(19) United States
(12) Patent Application Publication
(10) Pub. No.: US 2006/0104971 A1
(43) Pub. Date: May 18, 2006

(54) Multivalent lymphotoxin beta receptor agonists and therapeutic uses thereof

(75) Inventors: Ellen Garber, Cambridge, MA (US); Veronique Bailly, Boxborough, MA (US); Jeffrey L. Browning, Cambridge, MA (US)

Correspondence Address: LAHIVE & COCKFIELD, 28 STATE STREET, BOSTON, MA, 02109, US

(73) Assignee: Biogen Idec MA Inc., Cambridge, MA (US)

(21) Appl. No.: 11/155,444

(22) Filed: Jun. 17, 2005

Related U.S. Application Data
(63) Continuation of application No. PCT/US03/41393, filed on Dec. 22, 2003.
(60) Provisional application No. 60/435,154, filed on Dec. 20, 2002.

(51) Int. Cl. A61K 39/395; C07H 21/04; C12P 21/06; C12N 5/06; C07K 16/30

(52) U.S. Cl. 424/143.1; 435/69.1; 435/334; 435/320.1; 530/388.22; 536/23.53

(57) ABSTRACT

Multivalent antibody constructs that are specific for the human lymphotoxin beta receptor, as well as their use in treating cancer and inhibiting tumor volume in a subject are disclosed.

### RELATED APPLICATIONS

This application is a continuation of International Patent Application Serial No. PCT/US03/041393, filed on Dec. 22, 2003, which claims priority to U.S. Provisional Application No. 60/435,154, filed Dec. 20, 2002. This application is also related to U.S. Provisional Application No. 60/435,185, filed Dec. 20, 2002. The entire contents of each of these patents and patent applications are hereby incorporated herein by reference.

### FIELD OF THE INVENTION

This invention is in the fields of immunology and cancer diagnosis and therapy. More particularly it concerns the production and use of multivalent lymphotoxin beta receptor (LT-β-R) agonist antibody constructs in combination with chemotherapeutic agent(s) in therapeutic methods.

### BACKGROUND

Lymphotoxin beta receptor (LT-β-R) is a member of the tumor necrosis factor family, which has a well-described role both in the development of the immune system and in the functional maintenance of a number of cells in the immune system including follicular dendritic cells and a number of stromal cell types (Matsumoto et al., 1996 Immunol. Rev. 156: 137). Known ligands to the LT-β-R include LTα1/β2 and a second ligand called LIGHT (Mauri et al., 1998 Immunity 8: 21). Activation of LT-β-R, either by soluble ligands or agonistic antireceptor monoclonal antibodies has been found to induce the death of certain carcinomas (Browning, J. L. et al., (1996) J. Exp. Med. 183: 867-878 and U.S. Pat. No. 6,312,691).

The present invention provides multivalent LT-β-R agonists and therapeutic uses thereof.

### SUMMARY OF THE INVENTION

In one aspect, the present invention provides for multivalent antibody constructs that are human lymphotoxin-beta receptor (LT-β-R) agonists. In one embodiment, a multivalent antibody construct comprises at least one antigen recognition site specific for a LT-β-R epitope. In certain embodiments, at least one of the antigen recognition sites is located on a scFv domain, while in other embodiments, all antigen recognition sites are located on scFv domains.

The invention provides a multivalent antibody comprising at least one antigen recognition site specific for a lymphotoxin-beta receptor (LT-β-R) epitope. In one embodiment, at least one antigen recognition site is located within a scFv domain. In another embodiment, all antigen recognition sites are located within scFv domains. IN still another embodiment, the antibody construct is monospecific, including, for example, the epitope to which CBE11 binds. In still another embodiment, the multivalent antibody of the invention is tetravalent. In still another embodiment, the antibody construct is specific for the BHA10 epitope. In another embodiment of the invention, the antibody construct is bispecific. In one embodiment, the antibody construct has two CBE11-specific antigen recognition sites and two BHA10-specific recognition sites.

Antibody constructs may be bivalent, trivalent, tetravalent or pentavalent. In certain embodiments, the antibody construct is monospecific. In one embodiment, the antibody construct is specific for the epitope which CBE11 binds, and, in some embodiments, is tetravalent. In another embodiment, the antibody construct is specific for the epitope which BHA10 binds, and, in some embodiments, is tetravalent. In certain embodiments, at least one antigen recognition site is located within a scFv domain. In certain embodiments, all antigen recognition sites are located within scFv domains. Other antibody constructs may be multispecific for different epitopes on human LT-β receptors. In certain embodiments, the antibody construct is bispecific. In other embodiments, the antibody construct is specific for at least two members of the group of lymphotoxin-beta receptor (LT-β-R) epitopes consisting of: BKA11, CDH10, BCG6, AGH1, BDA8, CBE11 and BHA10. In one embodiment, the antibody construct is specific for the epitopes to which CBE11 and BHA10 bind, and in certain embodiments, is tetravalent. In one embodiment, the antibody construct has two CBE11-specific antigen recognition sites and two BHA10-specific recognition sites. In any of the multispecific antibody constructs, at least one antigen recognition site may be located on a scFv domain, and in certain embodiments, all antigen recognition sites are located on scFv domains.

The present invention further provides antibody constructs comprising SEQ ID NOs:1-10, as well as nucleic acids and vectors encoding the same, and host cells comprising the nucleic acids and vectors.

In another aspect, the present invention provides pharmaceutical compositions comprising the subject antibody constructs and a pharmaceutically acceptable carrier. In certain embodiments, the pharmaceutical compositions may further comprise an effective amount of a chemotherapeutic agent, wherein the administration of said composition to a subject results in supra-additive inhibition of a tumor. In another aspect, the present invention provides pharmaceutical delivery devices containing or able to be loaded with an effective amount of the subject multivalent antibody constructs and a pharmaceutically acceptable carrier. In certain embodiments, the pharmaceutical delivery device further contains or is able to be loaded with an effective amount of a chemotherapeutic agent, wherein the administration of the construct and the agent with said device results in supra-additive inhibition of a tumor.

In another aspect, the present invention provides methods for treating cancer in a subject, comprising administering to the subject an effective amount of a subject antibody construct. In certain embodiments, the subject is human. The present invention also provides methods of inhibiting tumor volume in a subject comprising the step of administering an effective amount of a subject antibody construct to the subject. In certain embodiments, the method of inhibiting tumor volume comprises administering an effective amount of the subject antibody constructs and a chemotherapeutic agent to the subject, wherein the administration of said construct and said agent results in supra-additive inhibition of the tumor.

The invention further provides kits including the subject pharmaceutical compositions or drug delivery devices, and optionally instructions for their use. Uses for such kits include, for example, therapeutic applications. In certain embodiments, the subject compositions contained in any kit have been lyophilized and require rehydration before use.

In one embodiment, the pharmaceutical composition comprising the multivalent antibody of the invention further comprises an effective amount of a chemotherapeutic agent, wherein the administration of said composition results in supra-additive inhibition of a tumor. The invention also describes pharmaceutical compositions comprising an effective amount of a multivalent antibody construct of the invention and a pharmaceutically acceptable carrier. In one embodiment, the pharmaceutical composition further comprises an effective amount of a chemotherapeutic agent, wherein the administration of said composition results in supra-additive inhibition of a tumor.

The invention includes a pharmaceutical delivery device containing or able to be loaded with an effective amount of a multivalent antibody construct of the invention, and a pharmaceutically acceptable carrier. In one embodiment, the pharmaceutical delivery device further contains or is able to be loaded with an effective amount of a chemotherapeutic agent, wherein the administration of said construct and said agent with said device results in supra-additive inhibition of a tumor.

The invention also includes a kit for treating cancer in a subject, comprising a composition comprising the multivalent antibody of the invention. In one embodiment, the kit also includes instructions. In another embodiment, the kit includes a chemotherapeutic agent.

The invention also describes a method of inhibiting tumor volume in a subject comprising the step of administering an effective amount of a multivalent antibody construct of the invention to said subject

Other features and advantages of the invention will be apparent from the following detailed description, and from the claims.

### BRIEF DESCRIPTION OF THE DRAWINGS

FIGS. 1A and 1B show the polynucleotide (SEQ ID NO: 1) and deduced polypeptide sequence (SEQ ID NO: 2), respectively, of the mature heavy chain of the huCBE11/huBHA10 Bispecific-1 antibody construct, respectively.

FIG. 2 shows the polynucleotide (SEQ ID NO: 3) and deduced polypeptide sequence (SEQ ID NO: 4) of the mature light chain of the huCBE11/huBHA10 Bispecific-1 antibody construct.

FIGS. 3A and 3B show the polynucleotide (SEQ ID NO: 5) and deduced polypeptide sequence (SEQ ID NO: 6), respectively, of the mature huCBE11/huBHA10 Bispecific-2 antibody construct.

FIGS. 4A and 4B show the polynucleotide (SEQ ID NO: 7) and deduced polypeptide sequence (SEQ ID NO: 8), respectively, of the mature heavy chain of the huCBE11 Monospecific-1 antibody construct.

FIGS. 5A and 5B show the polynucleotide (SEQ ID NO: 9) and deduced polypeptide sequence (SEQ ID NO: 10), respectively, of the mature huCBE11 Monospecific-2 antibody construct.

FIGS. 6A and 6B show schematics of the tertiary structure of the Bispecific antibody constructs with the variable domains indicated by the appropriately shaded regions. FIG. 6A shows the structure of the Bispecific-1 and Bispecific-2 antibodies which comprise CBE11 and BHA10 antigen recognition sites. FIG. 6B shows the structure of Monospecific-1 and Monospecific-2 tetravalent antibodies comprising CBE11 antigen recognition sites.

FIG. 7 depicts schematics of the tertiary structures of other antibody constructs that may be produced using the methods of the invention.

FIG. 8 depicts a graph showing HT29 cell proliferation as a function of antibody concentration for eight agonist anti-LT-beta-R antibodies, including the huCBE11/huBHA10 Bispecific-1 and Bispecific 2 antibody constructs (filled circles and filled squares, respectively), Monospecific-1 and Monospecific 2 antibody constructs (filled triangles and filled diamonds, respectively), the humanized antibody huCBE11 (open triangles), humanized antibody huBHA10 (open squares), humanized antibodies huCBE11 and huBHA10 administered in conjunction (open diamonds) and the pentameric chuCBE11 antibody (open circles).

FIG. 9 depicts a graph showing the response of the WiDr human colon adenocarcinoma tumor to Bispecific-1 as measured by the tumor weight observed at various days post-implantation for the indicated dosages of Bispecific-1 (triangles, open squares, open and closed circles), vehicle (PBS control, crosses), and taxol (closed squares). The first and last administrations of each dose are indicated by the arrows.

FIG. 10 depicts a retrospective comparison of activity of huCBE11 and Bispecific-1. with data from multiple tumor inhibition experiments. Data was calculated based upon % inhibition of tumor growth calculated by using the following formula (100−(100×(Test group mean/placebo control group mean))) on either day 34 or 35 on each study. Each data point on the graph represents one experimental test group from a study.

FIGS. 11A and 11B show the sequences comprising the pentameric form of CBE11 antibody. FIG. 11A depicts the polynucleotide (SEQ ID NO: 17) and deduced polypeptide sequence (SEQ ID NO: 18) of the mature pentameric chimeric CBE11 heavy chain. FIG. 11B depicts the polynucleotide (SEQ ID NO: 19) and deduced polypeptide sequence (SEQ ID NO: 20) of mature chimeric CBE11 light chain.

### DETAILED DESCRIPTION OF THE INVENTION

1. Definitions

For convenience, before further description of the present invention, certain terms employed in the specification, examples and appended claims are defined here. The singular forms “a”, “an”, and “the” include plural references unless the context clearly dictates otherwise.

The term “administering” includes any method of delivery of a compound of the present invention, including but not limited to, a pharmaceutical composition or therapeutic agent, into a subject's system or to a particular region in or on a subject. The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

As used herein, the term “antibody” is meant to refer to complete, intact antibodies, and Fab, Fab′, F(ab)₂, Fᵥ, and other fragments thereof. Complete, intact antibodies include, for example, monoclonal antibodies such as murine monoclonal antibodies, chimeric antibodies, anti-idiotypic antibodies, anti-anti-idiotypic antibodies, and humanized antibodies, as well as mutivalent forms thereof. The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. “Constant” domains on the light chain are referred to interchangeably as “light chain constant regions”, “light chain constant domains”, “CL” regions or “CL” domains). “Constant” domains on the heavy chain are referred to interchangeably as “heavy chain constant regions”, “heavy chain constant domains”, “CH” regions or “CH” domains). “Variable” domains on the light chain are referred to interchangeably as “light chain variable regions”, “light chain variable domains”, “VL” regions or “VL” domains). “Variable” domains on the heavy chain are referred to interchangeably as “heavy chain constant regions”, “heavy chain constant domains”, “CH” regions or “CH” domains).

The term “region” refers to a part or portion of an antibody chain and includes constant or variable domains as defined herein, as well as more discrete parts or portions of said domains. For example, light chain variable domains or regions include “complementarity determining regions” or “CDRs” interspersed among “framework regions” or “FRs”, as defined herein.

Immunoglobulins or antibodies can exist in monomeric or polymeric form. The term “antigen-binding fragment” refers to a polypeptide fragment of an immunoglobulin or antibody binds antigen or competes with intact antibody (i.e., with the intact antibody from which they were derived) for antigen binding (i.e., specific binding). The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

Binding fragments are produced by recombinant DNA techniques, or by enzymatic or chemical cleavage of intact immunoglobulins. Binding fragments include Fab, Fab′, F(ab′)₂, Fabc, Fv, single chains, and single-chain antibodies. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

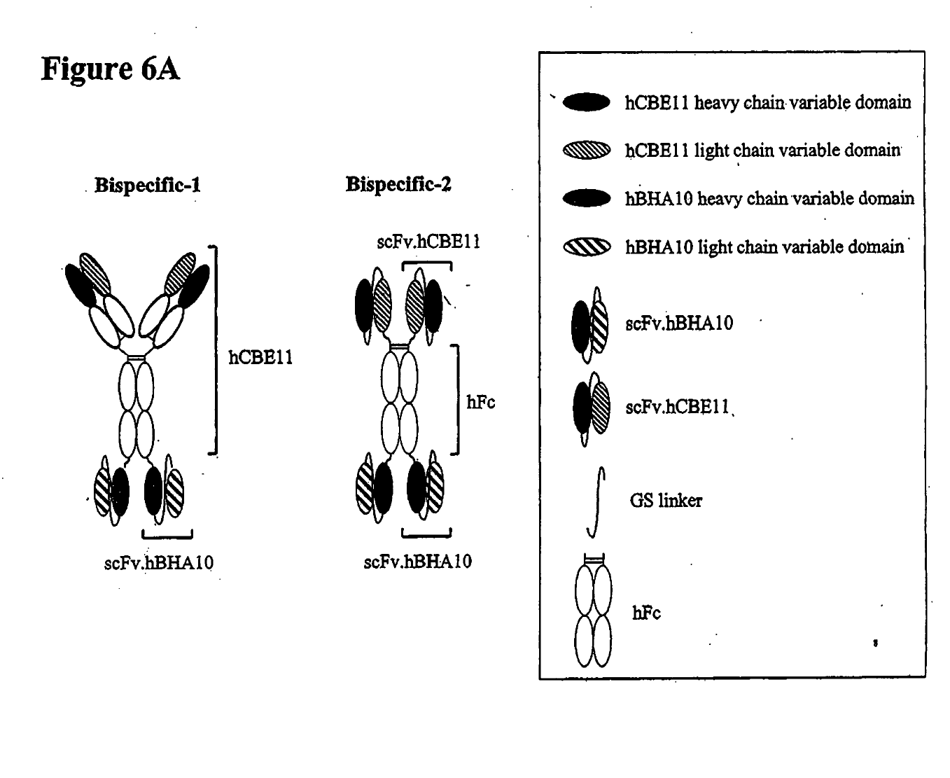

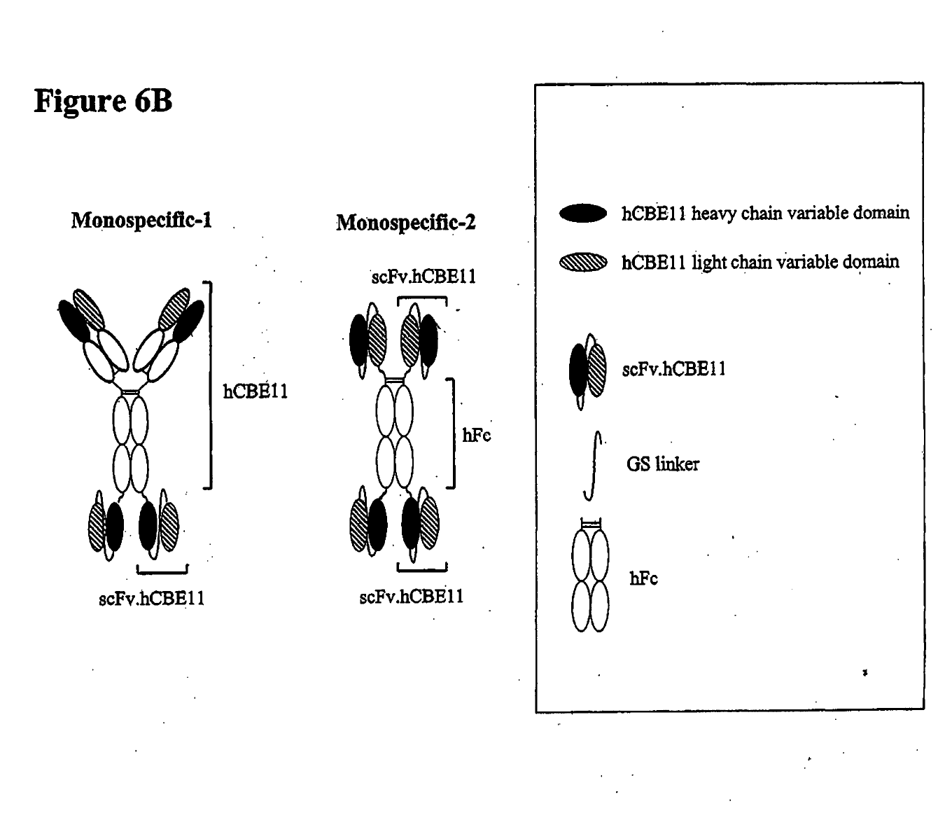

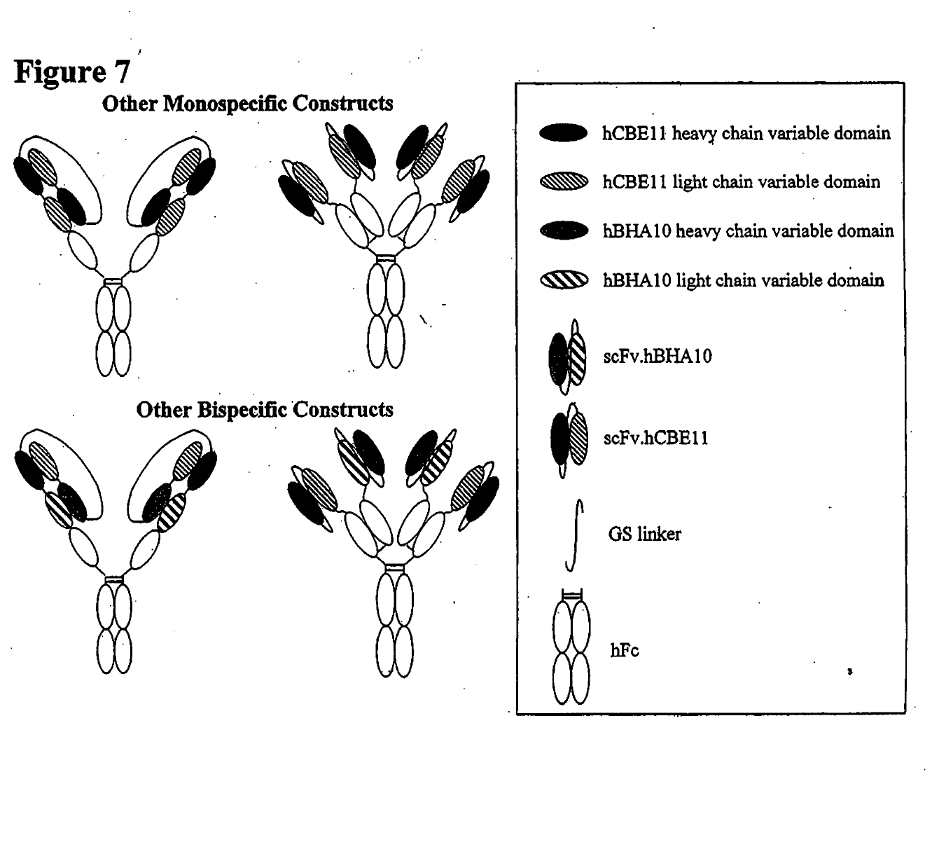

The term “antibody construct” refers to a recombinant molecule that comprises two or more antigen-binding fragments coming from the variable domains of the heavy chain and light chain of an antibody and may comprise the entire or part of the constant regions of an antibody from any of the five Ig classes (for example IgA, IgD, IgE, IgG and IgM). For example, the antibody construct may be made of an antibody which heavy chains comprise at their C-terminus a single chain variable fragment. In another example, the antibody construct may be made of the entire or part of the constant region of the two heavy chains of an antibody which comprise at their carboxy- and amino-termini a single chain variable fragment. Examples of each of these constructs is depicted schematically in FIG. 6. In yet another example, the antibody construct may comprise two heavy chains having two or more variable regions and two light chains having one or more variable regions where the two heavy chains are joined by a disulfide bond or other covalent linkage. In another example, the antibody construct may comprise two heavy chains comprising two or more variable regions where the two heavy chains are joined by a disulfide bond or other covalent linkage. Examples of each of these constructs is depicted schematically in FIG. 7. Other examples of antibody constructs of the invention are described in the Detailed Description of the Invention and Exemplification.

The term “antigen” as used herein, means a molecule which is reactive with a specific antibody.

The term “antigen binding site” or “antigen recognition site” refers to a region of an antibody that specifically binds an epitope on an antigen.

The term “cancer” or “neoplasia” refers in general to any malignant neoplasm or spontaneous growth or proliferation of cells. The term as used herein encompasses both fully developed malignant neoplasms, as well as premalignant lesions. A subject having “cancer”, for example, may have a tumor or a white blood cell proliferation such as leukemia. In certain embodiments, a subject having cancer is a subject having a tumor, such as a solid tumor. Cancers involving a solid tumor include but are not limited to non small cell lung cancer (NSCLC), testicular cancer, lung cancer, ovarian cancer, uterine cancer, cervical cancer,, pancreatic cancer, colorectal cancer (CRC), breast cancer, as well as on prostate, gastric, skin, stomach, esophagus and bladder cancer.

The term “chemotherapeutic agent” refers to any small molecule or composition used to treat disease caused by a foreign cell or malignant cell, such as a tumor cell. Non-limiting examples of chemotherapeutic agents include agents that disrupt DNA synthesis, are inhibitors of topoisomerase I, are alkylating agents, or are plant alkaloids. The term “agent that disrupts DNA synthesis” refers to any molecule or compound able to reduce or inhibit the process of DNA synthesis. Examples of agents that disrupt DNA synthesis include but are not limited to nucleoside analogs such as pyrimidine or purine analogs, including, for example but not limited to, gemcitabine or alternatively anthracycline compounds, including for example but not limited to, adriamycin, daunombicin, doxorubicin, and idambicin and epipodophyllotoxins such as etoposide and teniposide. The term “topoisomerase I inhibitor” refers to a molecule or compound that inhibits or reduces the biological activity of a topoisomerase I enzyme. Including for example, but not limited to, camptosar. The term “alkylating agent” refers to any molecule or compound able to react with the nucleophilic groups of (for examples, amines, alcohols, phenols, organic and inorganic acids) and thus add alkyl groups (for example, ethyl or methyl groups) to another molecule such as a protein or nucleic acid. Examples of alkylating agents used as chemotherapeutic agents include bisulfan, chlorambucil, cyclophosphamide, ifosfamide, mechlorethamine, melphalan, thiotepa, various nitrosourea compounds, and platinum compounds such as cisplatin and carboplatin. The term “plant alkaloid” refers a compound belonging to a family of alkaline, nitrogen-containing molecules derived from plants that are biologically active and cytotoxic. Examples of plant alkoids include, but are not limited to, taxanes such as taxol, docetaxel and paclitaxel and vincas such as vinblastine, vincristine, and vinorelbine.

The term “chimeric antibody” refers to an antibody whose light and heavy chain genes have been constructed, typically by genetic engineering, from immunoglobulin gene segments belonging to different species. For example, the variable (V) segments of the genes from a mouse monoclonal antibody may be joined to human constant (C) segments, such as IgG1 and IgG4. Human isotype IgG1 is preferred. A typical chimeric antibody is thus a hybrid protein consisting of the V or antigen-binding domain from a mouse antibody and the C or effector domain from a human antibody.

The term “effective amount” refers to that amount of a compound, material, or composition comprising a compound of the present invention which is sufficient to effect a desired result, including, but not limited to, for example, reducing tumor volume either in vitro or in vivo. An effective amount of a pharmaceutical composition of the present invention is an amount of the pharmaceutical composition that is sufficient to effect a desired clinical result, including but not limited to, for example, ameliorating, stabilizing, preventing or delaying the development of cancer in a patient. In either case, an effective amount of the compounds of the present invention can be administered in one or more administrations. Detection and measurement of these above indicators are known to those of skill in the art, including, but not limited for example, reduction in tumor burden, inhibition of tumor size, reduction in proliferation of secondary tumors, expression of genes in tumor tissue, presence of biomarkers, lymph node involvement, histologic grade, and nuclear grade.

The term “epitope” refers to the region of an antigen to which an antibody or antibody construct binds preferentially and specifically. A monoclonal antibody binds preferentially to a single specific epitope of a molecule that can be molecularly defined. In the present invention, multiple epitopes can be recognized by a multispecific antibody.

The term “inhibition of tumor volume” refers to any decrease or reduction in a tumor volume.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The term “lymphotoxin-beta receptor (LT-β-R) agonist” refers to any agent which can augment ligand binding to the LT-β-R, cell surface LT-β-R clustering and/or LT-β-R signaling.

The phrase “multivalent antibody” or “multivalent antibody construct” refers to an antibody or antibody construct comprising more than one antigen recognition site. For example, a “bivalent” antibody construct has two antigen recognition sites, whereas a “tetravalent” antibody construct has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody construct of the invention. For example, a “monospecific” antibody construct's antigen recognition sites all bind the same epitope. A “bispecific” antibody construct has at least one antigen recognition site that binds a first epitope and at least one antigen recognition site that binds a second epitope that is different from the first epitope. A “multivalent monospecific” antibody construct has multiple antigen recognition sites that all bind the same epitope. A “multivalent bispecific” antibody construct has multiple antigen recognition sites, some number of which bind a first epitope and some number of which bind a second epitope that is different from the first epitope. The terms “Bispecific-1” (also referred to as “BS-1”), “Bispecific-2”, (also referred to as “BS-2”), “Monospecific-1” (also referred to as “MS-1”), and “Monospecific-2” (also referred to as “MS-2”) refer to particular antibody constructs as further described herein. In one embodiment of the invention, the antibody is a monospecific tetravalent antibody, wherein the antibody comprises four CBE11 antigen recognition sites, as shown in FIG. 6B.

A “patient” or “subject” or “host” refers to either a human or non-human animal.

The term “pharmaceutical delivery device” refers to any device that may be used to administer a therapeutic agent or agents to a subject. Non-limiting examples of pharmaceutical delivery devices include hypodermic syringes, multichamber syringes, stents, catheters, transcutaneous patches, microneedles, microabraders, and implantable controlled release devices. In one embodiment, the term “pharmaceutical delivery device” refers to a dual-chambered syringe capable of mixing two compounds prior to injection.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically-acceptable salts” refers to the relatively non-toxic, inorganic and organic acid addition salts of compounds.

The term “Fv fragment” refers to the fragment of an antibody comprising the variable domains of its heavy chain and light chain. The term Fc fragment refers to the fragment of an antibody comprising the constant domain of its heavy chain.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

The term “supra-additive inhibition of a tumor” refers to a total decrease in tumor volume which is greater than the sum of the effects of a combination of agents due to each individual agent. In one embodiment, supra-additive inhibition includes a mean tumor inhibition produced by administration of a combination of a LT-β-R agonist and a chemotherapeutic agent, which is not a LT-β-R agonist, that is statistically signficantly higher than the sum of the tumor inhibition produced by the individual administration of either a LT-β-R agonist or chemotherapeutic agent alone. Whether tumor inhibition produced by combination administration of a LT-β-R agonist and a chemotherapeutic agent is “statistically significantly higher” than the expected additive value of the individual compounds may be determined by a variety of statistical methods as described in the Detailed Description of the Invention.

The term “synergistic” refers to a combination which is more effective than the additive effects of any two or more single agents. In one embodiment of the invention, the term synergistic includes a combination type of supra-additive inhibition in which both the LT-β-R agonist and chemotherapeutic agent individually have the ability to inhibit tumor volume. The term “potentiation” refers to a case in which simultaneous effect of two or more agents is greater than the sum of the independent effects of the agents. In a certain embodiment, potentiation refers to type of supra-additive inhibition in which only one of the LT-β-R agonist or chemotherapeutic agent individually have the ability to inhibit tumor volume.

“Treating” cancer in a subject or “treating” a subject having cancer refers to subjecting the subject to a pharmaceutical treatment, e.g., the administration of a drug, such that the extent of cancer is decreased or prevented. Treatment includes (but is not limited to) administration of a composition, such as a pharmaceutical composition, and may be performed either prophylactically, or subsequent to the initiation of a pathologic event.

The term “tumor volume” refers to the total size of the tumor, which includes the tumor itself plus affected lymph nodes if applicable. Tumor volume may be determined by a variety of methods known in the art, such as, e.g. by measuring the dimensions of the tumor using calipers, computed tomography (CT) or magnetic resonance imaging (MRI) scans, and calculating the volume using equations based on, for example, the z-axis diameter, or on standard shapes such as the sphere, ellipsoid, or cube.

2. Multivalent LT-β-R Agonist Antibody Constructs and Methods of Making the Same

In one embodiment, the multivalent antibody constructs of this invention are agonists of the lymphotoxin-beta receptor and comprise at least two domains that are capable of binding to the receptor and inducing LT-β-R signaling. These constructs can include a heavy chain containing two or more variable regions comprising antigen recognitions sites specific for binding the LT-beta receptor and a light chain containing one or more variable regions or can be constructed to comprise only heavy chains or light chains containing two or more variable regions comprising CDRs specific for binding the LT-beta receptor.

In one aspect, the present invention provides for multivalent antibody constructs that are human lymphotoxin-beta receptor (LT-β-R) agonists. In one embodiment, a multivalent antibody construct comprises at least one antigen recognition site specific for a LT-β-R epitope. In certain embodiments, at least one of the antigen recognition sites is located within a scFv domain, while in other embodiments, all antigen recognition sites are located within scFv domains.

Antibody constructs may be bivalent, trivalent, tetravalent or pentavalent. In certain embodiments, the antibody construct is monospecific. In one embodiment, the antibody construct is specific for the epitope to which CBE11 binds. In other embodiments, the antibody of the invention is a monospecific tetravalent LT-β-R agonist antibody comprising four CBE11-antigen recognition sites. In another embodiment, the antibody construct is specific for the BHA10 epitope, and, in some embodiments, is tetravalent. In any of these embodiments, at least one antigen recognition site may be located on a scFv domain, and in certain of these embodiments, all antigen recognition sites may be located on scFv domains. Antibodies may be multispecific, wherein the antibody of the invention binds to different epitopes on human LT-β receptors.

In certain embodiments, the antibody construct is bispecific. In other embodiments, the antibody construct is specific for at least two members of the group of lymphotoxin-beta receptor (LT-β-R) epitopes consisting of the epitopes to which one of following antibodies bind: BKA11, CDH10, BCG6, AGH1, BDA8, CBE11 and BHA10. In one embodiment, the antibody construct is specific for the epitope to which the CBE11 and BHA10 antibodies bind, and in certain embodiments, is tetravalent. In one embodiment, the antibody construct has two CBE11-specific antigen recognition sites and two BHA10-specific recognition sites, wherein the antibody is a bispecific tetravalent LT-β-R agonist antibody. In any of the multispecific antibody constructs, at least one antigen recognition site may be located on a scFv domain, and in certain embodiments, all antigen recognition sites are located on scFv domains.

In still other embodiments, the antibody constructs of the invention comprise the following polynucleotide sequences and encoded polypeptide sequences:

| Sequence | Figure | Description |
|---|---|---|
| SEQ ID NO: 1 | 1A | Polynucleotide sequence of mature heavy |
| | | chain of the huCBE11/huBHA10 |
| | | Bispecific-1 antibody construct |
| SEQ ID NO: 2 | 1B | Polypeptide sequence of mature heavy |
| | | chain of the huCBE11/huBHA10 |
| | | Bispecific-1 antibody construct |
| SEQ ID NO: 3 | 2 | Polynucleotide sequence of mature light |
| | | chain of the huCBE11/huBHA10 |
| | | Bispecific-1 antibody construct |
| SEQ ID NO: 4 | 2 | Polypeptide sequence of mature light |
| | | chain of the huCBE11/huBHA10 |
| | | Bispecific-1 antibody construct |
| SEQ ID NO: 5 | 3A | Polynucleotide sequence of mature |
| | | huCBE11/huBHA10 Bispecific-2 |
| | | antibody construct |
| SEQ ID NO: 6 | 3B | Polypeptide sequence of mature |
| | | huCBE11/huBHA10 Bispecific-2 |
| | | antibody construct |
| SEQ ID NO: 7 | 4A | Polynucleotide sequence of mature heavy |
| | | chain of the huCBE11 Monospecific-1 |
| | | antibody construct |
| SEQ ID NO: 8 | 4B | Polypeptide sequence of mature heavy |
| | | chain of the huCBE11 Monospecific-1 |
| | | antibody construct. |
| SEQ ID NO: 9 | 5A | Polynucleotide sequence of mature |
| | | huCBE11 Monospecific-2 antibody |
| | | construct |
| SEQ ID NO: 10 | 5B | Polypeptide sequence of mature huCBE11 |
| | | Monospecific-2 antibody construct |
| SEQ ID NO: 17 | 11A | Polynucleotide sequence of mature |
| | | CBE11 pentameric heavy chain antibody |
| | | construct |
| SEQ ID NO: 18 | 11A | Polypeptide sequence of mature CBE11 |
| | | pentameric heavy chain antibody |
| | | construct |
| SEQ ID NO: 19 | 11B | Polynucleotide sequence of mature |
| | | CBE11 chimeric light chain antibody |
| | | construct |
| SEQ ID NO: 20 | 11B | Polypeptide sequence of mature CBE11 |
| | | chimeric light chain antibody construct |

Examples 1-9 provide a detailed description for obtaining the bispecific, monospecific and pentameric LT-β-R agonist antibody constructs listed in the above table. Schematics for certain of these constructs are depicted in FIGS. 6A and 6B. However other LT-β-R agonist antibody constructs of the invention may be constructed using methods known in the art, as described briefly below. Several examples of such constructs are depicted in FIG. 7.

The antigen recogntion sites or entire variable regions may be derived from one or more parental antibodies. The parental antibodies can include naturally occurring antibodies or antibody fragments, antibodies or antibody fragments adapted from naturally occurring antibodies, antibodies constructed de novo using sequences of antibodies or antibody fragments known to be specific for the LT-beta receptor. Sequences that may be derived from parental antibodies include heavy and/or light chain variable regions and/or CDRs, framework regions or other portions thereof.

Multivalent, multispecific antibodies may contain a heavy chain comprising two or more variable regions and/or a light chain comprising one or more variable regions wherein at least two of the variable regions recognize different epitopes on the LT-beta receptor.

Multivalent, anti-LT-β-R antibodies may be constructed in a variety different ways using a variety of different sequences derived from parental anti-LT-β-R antibodies, including murine or humanized BHA10 (Browning et al., J. Immunol. 154: 33 (1995); Browning et al. J. Exp. Med. 183:867 (1996)) and/or murine or humanized CBE11 (U.S. Pat. No. 6,312,691).

The following hybridoma cell lines producing monoclonal anti-LT-β-R antibodies may be used to produce anti-LT-β-R antibodies from which to derive antibody construct sequences, which have been previously deposited with the American Type Culture Collection (ATCC) according to the provisions of the Budapest Treaty and have been assigned the indicated ATCC accession numbers:

| Cell Line | mAb Name | Accession No. |
|---|---|---|
| a) AG.H1.5.1 | AGH1 | HB 11796 |
| b) BD.A8.AB9 | BDA8 | HB 11798 |
| c) BC.G6.AF5 | BCG6 | B 11794 |
| d) BH.A10 | BHA10 | B 11795 |
| e) BK.A11.AC10 | BKA11 | B 11799 |
| f) CB.E11.1 | CBE11 | B 11793 |
| g) CD.H10.1 | CDH10 | B 11797 |

However, a variety of other multivalent antibody constructs may be developed by one of skill in the art using routine recombinant DNA techniques, for example as described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; Beidler et al. (1988) J. Immunol. 141:4053-4060; and Winter and Milstein, Nature, 349, pp. 293-99 (1991)). Preferably non-human antibodies are “humanized” by linking the non-human antigen binding domain with a human constant domain (e.g. Cabilly et al., U.S. Pat. No. 4,816,567; Morrison et al., Proc. Natl. Acad. Sci. U.S.A., 81, pp. 6851-55 (1984)).

Other methods which may be used to prepare the subject anti-LT-β-R antibody constructs are described in the following publications: Ghetie, Maria-Ana et al. (2001) Blood 97:1392-1398; Wolff, Edith A. et al. (1993) Cancer Research 53:2560-2565; Ghetie, Maria-Ana et al. (1997) Proc. Natl. Acad. Sci. 94:7509-7514; Kim, J. C. et al. (2002) Int. J. Cancer 97(4):542-547; Todorovska, Aneta et al. (2001) Journal of Immunological Methods 248:47-66; Coloma M. J. et al. (1997) Nature Biotechnology 15:159-163; Zuo, Zhuang et al. (2000) Protein Engineering (Suppl.) 13(5):361-367; Santos A. D., et al. (1999) Clinical Cancer Research 5:3118s-3123s; Presta, Leonard G. (2002) Current Pharmaceutical Biotechnology 3:237-256; van Spriel, Annemiek et al., (2000) Review Immunology Today 21(8) 391-397.

Candidate antibody constructs may be screened for activity using a variety of known assays. For example, screening assays to determine binding specificity are well known and routinely practiced in the art. For a comprehensive discussion of such assays, see Harlow et al. (Eds.), ANTIBODIES: A LABORATORY MANUAL; Cold Spring Harbor Laboratory; Cold Spring Harbor, N.Y., 1988, Chapter 6. The following Examples provide assays for determining the efficacy of LT-β-R activation by canditate LT-β-R agonist antibody constructs.

The LT-β-R agonist antibody constructs produced as described above may be purified to a suitable purity for use as a pharmaceutical composition. Generally, a purified composition will have one species that comprises more than about 85 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification, for example, immunoaffinity chromatography, size exclusion chromatography, etc. in light of the teachings herein. Purity of a polypeptide may be determined by a number of methods known to those of skill in the art, including for example, amino-terminal amino acid sequence analysis, gel electrophoresis and mass-spectrometry analysis.

In some embodiments, the multivalent antibodies and antibody fragments of the invention may be chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat conditions that may be alleviated or modulated by administration of the antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In other embodiments of the invention the antibodies or antigen-binding fragments thereof are conjugated to albumen using art recognized techniques.

In another embodiment of the invention, multivalent antibodies, or fragments thereof, are modified to reduce or eliminate potential glycosylation sites. Such modified antibodies are often referred to as “aglycosylated” antibodies. In order to improve the binding affinity of an antibody or antigen-binding fragment thereof, glycosylation sites of the antibody can be altered, for example, by mutagenesis (e.g., site-directed mutagenesis). “Glycosylation sites” refer to amino acid residues which are recognized by a eukaryotic cell as locations for the attachment of sugar residues. The amino acids where carbohydrate, such as oligosaccharide, is attached are typically asparagine (N-linkage), serine (O-linkage), and threonine (O-linkage) residues. In order to identify potential glycosylation sites within an antibody or antigen-binding fragment, the sequence of the antibody is examined, for example, by using publicly available databases such as the website provided by the Center for Biological Sequence Analysis (see http://www.cbs.dtu.dk/services/NetNGlyc/ for predicting N-linked glycoslyation sites) and http://www.cbs.dtu.dk/services/NetOGlyc/ for predicting O-linked glycoslyation sites). Additional methods for altering glycosylation sites of antibodies are described in U.S. Pat. Nos. 6,350,861 and 5,714,350.

In yet another embodiment of the invention, multivalent antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor (FcR), the immunoglobulin constant region segment of the antibody can be mutated at particular regions necessary for FcR interactions (see e.g., Canfield et al (1991) J. Exp. Med. 173:1483; and Lund, J. et al. (1991) J. of Immunol. 147:2657). Reduction in FcR binding ability of the antibody may also reduce other effector functions which rely on FcR interactions, such as opsonization and phagocytosis and antigen-dependent cellular cytotoxicity.

In a particular embodiment the invention further features multivalent antibodies having altered effector function, such as the ability to bind effector molecules, for example, complement or a receptor on an effector cell. In particular, the humanized antibodies of the invention have an altered constant region, e.g., Fc region, wherein at least one amino acid residue in the Fc region has been replaced with a different residue or side chain thereby reducing the ability of the antibody to bind the FcR. Reduction in FcR binding ability of the antibody may also reduce other effector functions which rely on FcR interactions, such as opsonization and phagocytosis and antigen-dependent cellular cytotoxicity. In one embodiment, the modified humanized antibody is of the IgG class, comprises at least one amino acid residue replacement in the Fc region such that the humanized antibody has an altered effector function, e.g., as compared with an unmodified humanized antibody. In particular embodiments, the humanized antibody of the invention has an altered effector function such that it is less immunogenic (e.g., does not provoke undesired effector cell activity, lysis, or complement binding), and/or has a more desirable half-life while retaining specificity for LTβR.

Alternatively, the invention features multivalent humanized antibodies having altered constant regions to enhance FcR binding, e.g., FcγR3 binding. Such antibodies are useful for modulating effector cell function, e.g., for increasing ADCC activity, e.g., particularly for use in oncology applications of the invention.

As used herein, “antibody-dependent cell-mediated cytotoxicity” and “ADCC” refer to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRIII and FcγRIII. of the antibody, e.g., a conjugate of the antibody and another agent or antibody.

In still another embodiment, the multivalent anti-LT-β-R antibodies of the invention can be conjugated to a chemotherapeutic agent to inhibit tumor volume in a supra-additive manner. Exemplary chemotherapeutics that can be conjugated to the antibodies of the present invention include, but are not limited to radioconjugates (90Y, 131I, 99mTc, 111In, 186Rh, et al.), tumor-activated prodrugs (maytansinoids, CC-1065 analogs, clicheamicin derivatives, anthracyclines, vinca alkaloids, et al.), ricin, diptheria toxin, pseudomonas exotoxin.

3. Combination Therapeutics Comprising the Use of Multivalent LT-β-R Agonist Antibody Constructs

The invention further provides for the use of a multivalent LT-β-R agonist antibody in combination with a chemotherapeutic agent to treat cancer, and/or inhibit tumor growth. Likewise, any of a variety of chemotherapeutic agents may be used or tested for use in the methods of the invention, provided that the combination of the agonist and agent acheives inhibition of a tumor greater than that expected by the simple addition of the effects of the agonist and agent alone. Such chemotherapeutic agents may include anti-metabolic agents, alkylating agents, platinum-based agents, anthracyclines, antibiotic agents, topoisomerase inhibitors, and others. Various forms of the chemotherapeutic agents and/or other biologically active agents may be used. These include, without limitation, such forms as uncharged molecules, molecular complexes, salts, ethers, esters, amides, and the like, which are biologically activated when implanted, injected or otherwise inserted into the tumor.

Chemotherapy drugs which can be used in combination with the multivalent antibodies of the invention can be divided into several categories based on how they affect specific chemical substances within cancer cells, which cellular activities or processes the drug interferes with, and which specific phases of the cell cycle the drug affects.

In certain embodiments, the chemotherapeutic agent is an agent that disrupts DNA synthesis. In one embodiment, the agent that disrupts DNA synthesis is a nucleoside analog compound. In certain embodiments, the nucleoside analog compound is gemcitabine. In another embodiment, the agent that disrupts DNA synthesis is a anthracycline compound, and in certain embodiments, the anthracycline compound is adriamycin.

In other embodiments, the chemotherapeutic agent is a topoisomerase I inhibitor. In certain embodiments, the topoisomerase I inhibitor is Camptosar.

The chemotherapeutic agent in other embodiments may be an alkylating agent. Alkylating agents work directly on DNA to prevent the cancer cell from reproducing. As a class of drugs, these agents are not phase-specific (in other words, they work in all phases of the cell cycle). Alkylating agents are commonly active against chronic leukemias, non-Hodgkin's lymphoma, Hodgkin's disease, multiple myeloma, and certain cancers of the lung, breast, and ovary. Examples of alkylating agents include busulfan, cisplatin, carboplatin, chlorambucil, cyclophosphamide, ifosfamide, dacarbazine (DTIC), mechlorethamine (nitrogen mustard), and melphalan. In one embodiment, the alkylating agent is a platinum compound, and in certain embodiments may be selected from the group consisting of carboplatin and cisplatin. In certain embodiments, the platinum compound is cisplatin.

In still other embodiments, the chemotherapeutic agent may be a plant alkaloid. In one embodiment, the plant alkaloid is a taxane, and in certain embodiments may be Taxol.

The multivalent antibody of the invention can be used in combination with a chemotherapeutic agent to treat cancer, wherein the combination of the chemotherapeutioc agent and the multivalent antibody has a supra-additive effect. As used herein, “supra-additive inhibition of a tumor” refers to mean tumor inhibition produced by administration of a combination of a LT-β-R agonist and a chemotherapeutic agent that is statistically signficantly higher than the sum of the tumor inhibition produced by the individual administration of either a LT-β-R agonist or chemotherapeutic agent alone. Whether tumor inhibition produced by combination administration of a LT-β-R agonist and a chemotherapeutic agent is “statistically significantly higher” than the expected additive value of the individual compounds may be determined by as follows. Such supra-additive inhibition may be potentiated, or synergistic, as defined above.

In general, supra-additive inhibition may be assessed by determining whether the combination treatment produces a mean tumor volume decrease in a treatment group that is statistically significantly supra-additive when compared to the sum of the mean tumor volume decreases produced by the individual treatments in their treatment groups respectively. The mean tumor volume decrease may be calculated as the difference between control group and treatment group mean tumor volume. The fractional inhibition of tumor volume, “fraction affected” (Fa), may be calculated by dividing the treatment group mean tumor volume decrease by control group mean tumor volume. An Fa of 1.000 indicates complete inhibition of the tumor. Testing for statistically significant potentiation requires the calculation of Fa for each treatment group. The expected additive Fa for a combination treatment was taken to be the sum of mean Fas from groups receiving either element of the combination. The Two-Tailed One-Sample T-Test, for example, may be used to evaluate how likely it is that the result obtained by the experiment is due to chance alone, as measured by the p-value. A p-value of less than 0.05 is considered statistically significant, including but not limited to between about 0.05 to about 0.04; between about 0.04 to about 0.03; between about 0.03 to about 0.02; between about 0.02 to about 0.01, that is, not likely to be due to chance alone. In certan cases, the p-value may be less than 0.01. Thus, Fa for the combination treatment group must be statistically significantly higher than the expected additive Fa for the single element treatment groups to deem the combination as resulting in a potentiated supra-additive effect.

Whether or not a synergistic effect results from a combination treatment may be evalued by the median-effect/combination-index isobologram method (Chou et al. (1984) Ad. Enzyme Reg. 22:27). In this method, combination index (CI) values are calculated for different dose-effect levels based on parameters dervied from median-effect plots of the LT-β-R agonist alone, the chemotherapeutic agent alone, and the combination of the two at fixed molar ratios. CI values of <1 indicate synergy, including but not limited to between about 0.85 to about 0.90; between about 0.70 to about 0.85; between about 0.30 to about 0.70; between about 0.10 to about 0.30. In yet another embodiment the combination index is less than 0.10. This analysis is preferably performed using CalcuSyn, Windows® Software for Dose Effect Analysis (Biosoft, Cambridge UK).

Any method known or later developed in the art for analyzing whether or not a supra-additive effect exists for a combination therapy is contemplated for use in screening for suitable chemotherapeutic agents.

Methods for testing candidate LT-β-R agonists in combination with chemotherapeutic agents in order to determine whether or not supra-additive inhibition of a tumor will occur are taught in Applicants' co-pending PCT Application entitled, “Novel Combination Therapeutics for Cancer”, filed on even date herewith, which is hereby incorporated by reference in its entirety, and in Provisional Application No. 60/435,185, filed Dec. 20, 2002.

4. Pharmaceutical Compositions

The invention provides pharmaceutical compositions comprising the above-described LT-β-R agonist antibody constructs. In certain embodiments, the pharmaceutical compostions may further comprise a chemotherapeutic agent. In one aspect, the present invention provides pharmaceutically acceptable compositions which comprise a therapeutically-effective amount of one or more of the compounds described above, formulated together with one or more pharmaceutically acceptable carriers (additives) and/or diluents. In another aspect, certain embodiments, the compounds of the invention may be administered as such or in admixtures with pharmaceutically acceptable carriers and may also be administered in conjunction with other chemotherapeutic agents. Conjunctive (combination) therapy thus includes sequential, simultaneous and separate, or co-administration of the active compound in a way that the therapeutical effects of the first administered one is not entirely disappeared when the subsequent is administered.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art. While it is possible for a compound of the present invention to be administered alone, it is preferable to administer the compound as a pharmaceutical formulation (composition). The compounds according to the invention may be formulated for administration in any convenient way for use in human or veterinary medicine, by analogy with other pharmaceuticals.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In one embodiment, the pharmaceutical compositions are formulated for parenteral administration. In one embodiment, the pharmaceutical composition is formulated for intraarterial injection. In another embodiment, the pharmaceutical compositions are formulated for systemic administration.

In other cases, the compounds of the present invention may contain one or more acidic functional groups and, thus, are capable of forming pharmaceutically-acceptable salts with pharmaceutically-acceptable bases.

Wetting agents, emulsifiers and lubricants, such as sodium lauryl sulfate and magnesium stearate, as well as coloring agents, release agents, coating agents, sweetening, flavoring and perfuming agents, preservatives and antioxidants may also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which may be combined with a carrier material to produce a single dosage form will vary depending upon the host being treated, the particular mode of administration. The amount of active ingredient which may be combined with a carrier material to produce a single dosage form will generally be that amount of the compound which produces a therapeutic effect.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions may also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which may be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which may be used include polymeric substances and waxes. The active ingredient may also be in micro-encapsulated form, if appropriate, with one or more of the above-described excipients.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

5. Delivery Methods and Devices

The pharmaceutical compositions of this invention may also be administered using a variety of pharmaceutical delivery devices may, which may include hypodermic syringes, multichamber syringes, stents, catheters, transcutaneous patches, microneedles, microabraders, and implantable controlled release devices. In one embodiment, a pharmaceutical delivery device contains or is able to be loaded with at least an effective amount of a LT-β-R agonist antibody construct. Such devices may have the ability to reconstitute a lyophilized form of the antibody construct in the device before delivery. In some embodiments, pharmaceutical delivery device contains or is able to be loaded with at least an effective amount of a LT-β-R agonist and an effective amount of a chemotherapeutic agent. The device may in some embodiments be able to deliver or administer the LT-β-R agonist antibody construct and chemotherapeutic agent simultaneously. The device may have the ability to mix the antibody construct and chemotherapeutic agent prior to administration with the device. In still other embodiments, the device may be able to administer the agonist antibody construct and chemotherapeutic agent consecutively.

One pharmaceutical delivery device is a multi-chambered syringe capable of mixing two compounds prior to injection, or delivering them sequentially. A typical dual-chamber syringe and a process for automated manufacture of prefilled such syringes is disclosed in Neue Verpackung, No.3, 1988, p. 50-52; Drugs Made in Germany, Vol. 30, Pag. 136-140 (1987); Pharm. Ind. 46, Nr. 10 (1984) p. 1045-1048 and Pharm. Ind. 46, Nr. 3 (1984) p. 317-318. The syringe type ampoule is a dual chamber device with a front bottle type opening for needle attachment, two pistons and an exterior type by-pass for mixing a lyophilized powder in the front chamber with a reconstitution liquid in the rear chamber. The process described includes the main steps of washing and siliconizing the syringe barrels, insertion of multiple barrels in carrier trays, sterilization, introduction of middle piston through barrel rear end, turning the trays upside down, introduction of the powder solution through the front opening, lyophilization to dry powder, closure of front opening while in the lyophilizing chamber, turning of trays, introduction of the reconstitution liquid through barrel rear end, insertion of rear piston, removal of products from trays and final control and packaging. Ampoules prefilled with the various components may be manufactured for use with the syringes.

In amother embodiment, the multichamber syringe is a Lyo-ject system (Vetter Pharma Turm, Yardley, Pa.). The Lyo-Ject allows the user to lyophilize the drug directly in a syringe, which is packaged with the diluent for quick reconstitution and injection. It is described in U.S. Pat. Nos. 4,874,381 and 5,080,649.

In other embodiments, the compounds are administered using two separate syringes, catheters, microneedles, or other device capable of accomplishing injection.

The pharmaceutical compositions of this invention may also be administered using microspheres, liposomes, other microparticulate delivery systems or sustained release formulations placed in, near, or otherwise in communication with affected tissues or the bloodstream. Suitable examples of sustained release carriers include semipermeable polymer matrices in the form of shaped articles such as suppositories or microcapsules. Implantable or microcapsular sustained release matrices include polylactides (U.S. Pat. No. 3,773,319; EP 58,481), copolymers of L-glutamic acid and gamma ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)).

The compositions of this invention will be administered at an effective dose to treat the particular clinical condition addressed. Determination of a preferred pharmaceutical formulation and a therapeutically efficient dose regimen for a given application is well within the skill of the art taking into consideration, for example, the condition and weight of the patient, the extent of desired treatment and the tolerance of the patient for the treatment.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

6. Therapeutic Methods

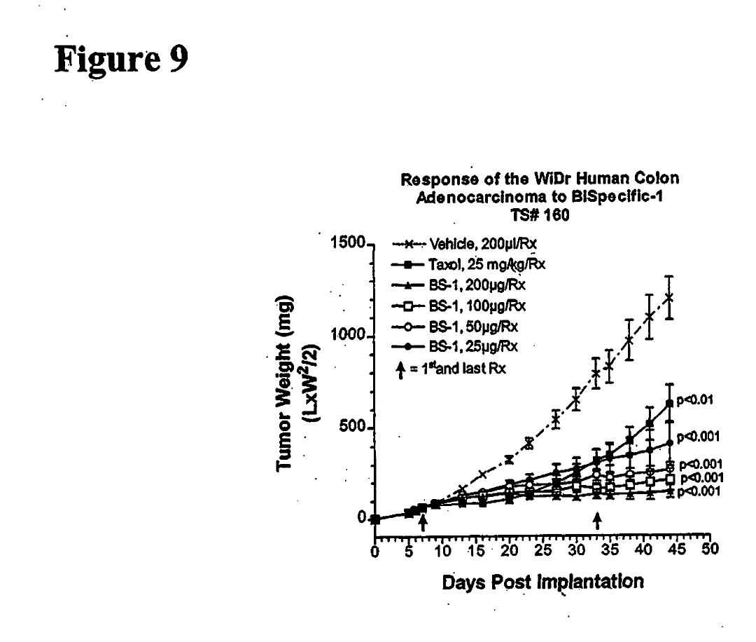

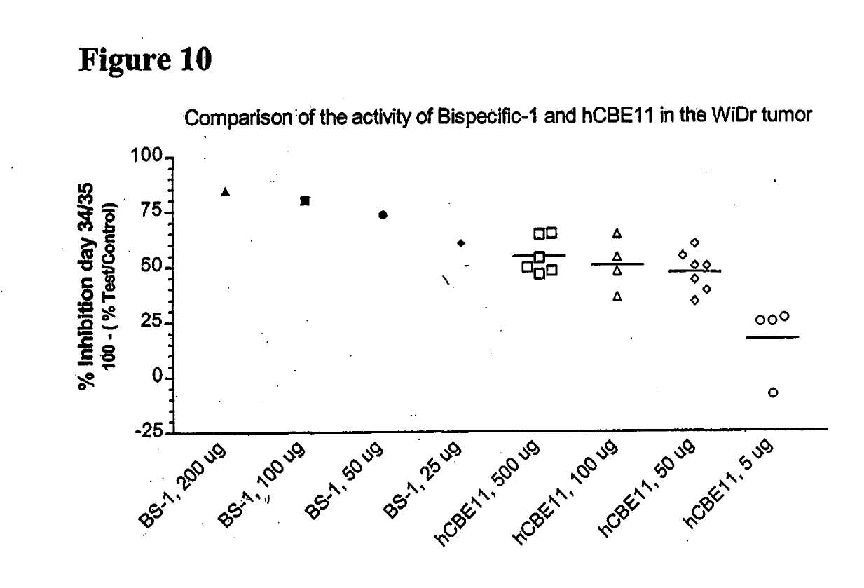

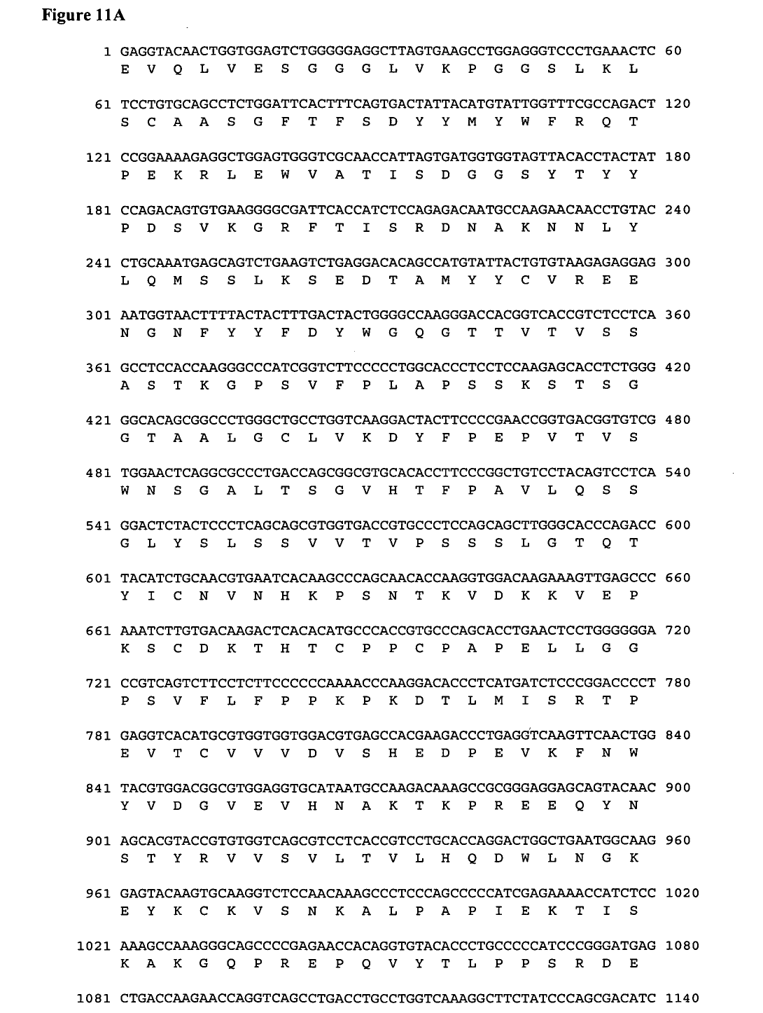

As described in Example 9 and as shown in FIGS. 9 and 10, the multivalent antibody constructs of the present invention are effective in significantly reducing tumor weight in vivo.

Hence, the present invention further provides novel therapeutic methods of treating cancer comprising administering to the subject an effective amount of a pharmaceutical composition, optionally using a delivery device described above. The methods of the present invention may be used to treat any cancer, including but not limited to treating solid tumors. Examples of solid tumors that can be treated by compounds of the present invention, include but are not limited to breast, testicular, lung, ovary, uterine, cervical, pancreatic, non small cell lung (NSCLC), colon, as well as on prostate, gastric, skin, stomach, esophagus and bladder cancer. In certain embodiments, the method comprises parenterally administering an effective amount of a subject pharmaceutical composition to a subject. In one embodiment, the method comprises intraarterial administration of a subject composition to a subject. In other embodiments, the method comprises administering an effective amount of a subject composition directly to the arterial blood supply of a tumor in a subject. In one embodiment, the methods comprises administering an effective amount of a subject composition directly to the arterial blood supply of the cancerous tumor using a catheter. In embodiments where a catheter is used to administer a subject composition, the insertion of the catheter may be guided or observed by fluoroscopy or other method known in the art by which catheter insertion may be observed and/or guided. In another embodiment, the method comprises chemoembolization. For example a chemoembolization method may comprise blocking a vessel feeding the cancerous tumor with a composition comprised of a resin-like material mixed with an oil base (e.g., polyvinyl alcohol in Ethiodol) and one or more chemotherapeutic agents. In still other embodiments, the method comprises systemic administration of a subject composition to a subject.

In general, chemoembolization or direct intraarterial or intravenous injection therapy utilizing pharmaceutical compositions of the present invention is typically performed in a similar manner, regardless of the site. Briefly, angiography (a road map of the blood vessels), or more specifically in certain embodiments, arteriography, of the area to be embolized may be first performed by injecting radiopaque contrast through a catheter inserted into an artery or vein (depending on the site to be embolized or injected) as an X-ray is taken. The catheter may be inserted either percutaneously or by surgery. The blood vessel may be then embolized by refluxing pharmaceutical compositions of the present invention through the catheter, until flow is observed to cease. Occlusion may be confirmed by repeating the angiogram. In embodiments where direct injection is used, the blood vessel is then infused with a pharmaceutical composition of the invention in the desired dose.

Embolization therapy generally results in the distribution of compositions containing inhibitors throughout the interstices of the tumor or vascular mass to be treated. The physical bulk of the embolic particles clogging the arterial lumen results in the occlusion of the blood supply. In addition to this effect, the presence of an anti-angiogenic factor(s) prevents the formation of new blood vessels to supply the tumor or vascular mass, enhancing the devitalizing effect of cutting off the blood supply. Direct intrarterial or intravenous generally results in distribution of compositions containing inhibitors throughout the interstices of the tumor or vascular mass to be treated as well. However, the blood supply is not generally expected to become occluded with this method.

Within one aspect of the present invention, primary and secondary tumors of the liver or other tissues may be treated utilizing embolization or direct intraarterial or intravenous injection therapy. Briefly, a catheter is inserted via the femoral or brachial artery and advanced into the hepatic artery by steering it through the arterial system under fluoroscopic guidance. The catheter is advanced into the hepatic arterial tree as far as necessary to allow complete blockage of the blood vessels supplying the tumor(s), while sparing as many of the arterial branches supplying normal structures as possible. Ideally this will be a segmental branch of the hepatic artery, but it could be that the entire hepatic artery distal to the origin of the gastroduodenal artery, or even multiple separate arteries, will need to be blocked depending on the extent of tumor and its individual blood supply. Once the desired catheter position is achieved, the artery is embolized by injecting compositions (as described above) through the arterial catheter until flow in the artery to be blocked ceases, preferably even after observation for 5 minutes. Occlusion of the artery may be confirmed by injecting radio-opaque contrast through the catheter and demonstrating by fluoroscopy or X-ray film that the vessel which previously filled with contrast no longer does so. In embodiments where direct injection is used, the artery is infused by injecting compositions (as described above) through the arterial catheter in a desired dose. The same procedure may be repeated with each feeding artery to be occluded.

In most embodiments, the subject pharmaceutical compositions will incorporate the substance or substances to be delivered in an amount sufficient to deliver to a patient a therapeutically effective amount of an incorporated therapeutic agent or other material as part of a prophylactic or therapeutic treatment. The desired concentration of active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art. The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

Dosage may be based on the amount of the composition per kg body weight of the patient. Other amounts will be known to those of skill in the art and readily determined. Alternatively, the dosage of the subject invention may be determined by reference to the plasma concentrations of the composition. For example, the maximum plasma concentration (Cmax) and the area under the plasma concentration-time curve from time 0 to infinity (AUC (0-4)) may be used. Dosages for the present invention include those that produce the above values for Cmax and AUC (0-4) and other dosages resulting in larger or smaller values for those parameters.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The combined use of several compounds of the present invention, or alternatively other chemotherapeutic agents, may reduce the required dosage for any individual component because the onset and duration of effect of the different components may be complimentary. In such combined therapy, the different active agents may be delivered together or separately, and simultaneously or at different times within the day. Toxicity and therapeutic efficacy of subject compounds may be determined by standard pharmaceutical procedures in cell cultures or experimental animals, e.g., for determining the LD50 and the ED50. Compositions that exhibit large therapeutic indices are preferred. Although compounds that exhibit toxic side effects may be used, care should be taken to design a delivery system that targets the compounds to the desired site in order to reduce side effects.

The data obtained from the cell culture assays and animal studies may be used in formulating a range of dosage for use in humans. The dosage of any supplement, or alternatively of any components therein, lies preferably within a range of circulating concentrations that include the ED50 with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

7. Kits

The present invention provides kits for treating various cancers. For example, a kit may comprise one or more pharmaceutical composition as described above and optionally instructions for their use. In still other embodiments, the invention provides kits comprising one more more pharmaceutical composition and one or more devices for accomplishing administration of such compositions. For example, a subject kit may comprise a pharmaceutical composition and catheter for accomplishing direct intraarterial injection of the composition into a cancerous tumor. In other embodiments, a subject kit may comprise pre-filled ampoules of an LT-β-R agonist antibody construct, optionally formulated as a pharmaceutical, or lyophilized, for use with a delivery device.

### EXEMPLIFICATION

The invention now being generally described, it will be more readily understood by reference to the following examples which are included merely for purposes of illustration of certain aspects and embodiments of the present invention, and are not intended to limit the invention in any way.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Production of muBHA10 and muCBE11 variable regions, murine-human BHA10 and CBE11 chimeric antibodies, reshaped BHA10 and CBE11 variable domains, expression vectors encoding huBHA10 and huCBE11, pentameric chCBE11 antibodies, and methods of purifiying and assaying the same have been previously described in Applicants' copending applications PCT publication no. WO 96/22788, PCT publication WO 02/30986, and PCT application no. PCT/US03/20762, which are each hereby incorporated by reference in their entirety.

### Example 1

### Construction and Expression of huCBE11/huBHA10 Bispecific-1 Antibody

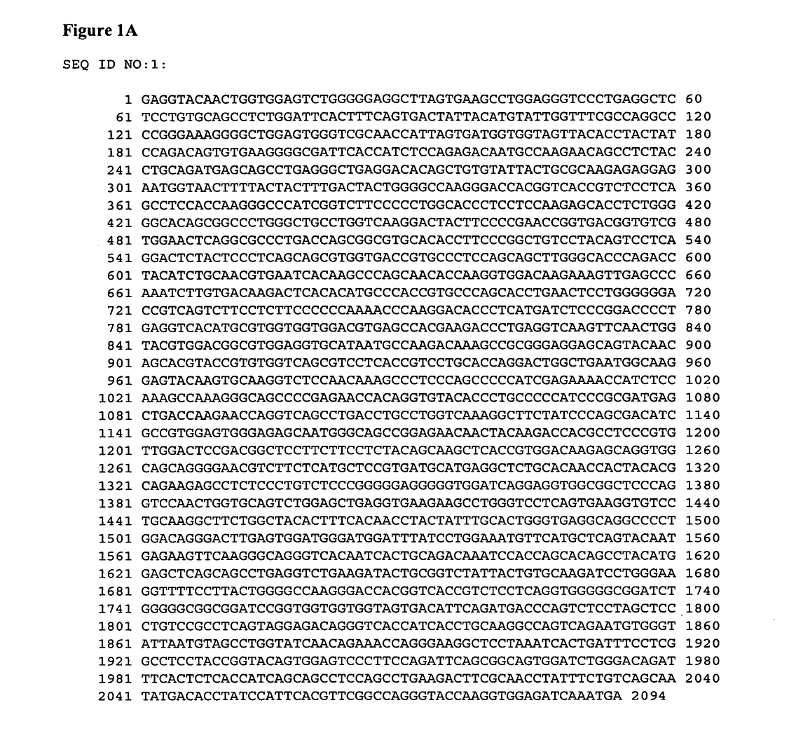

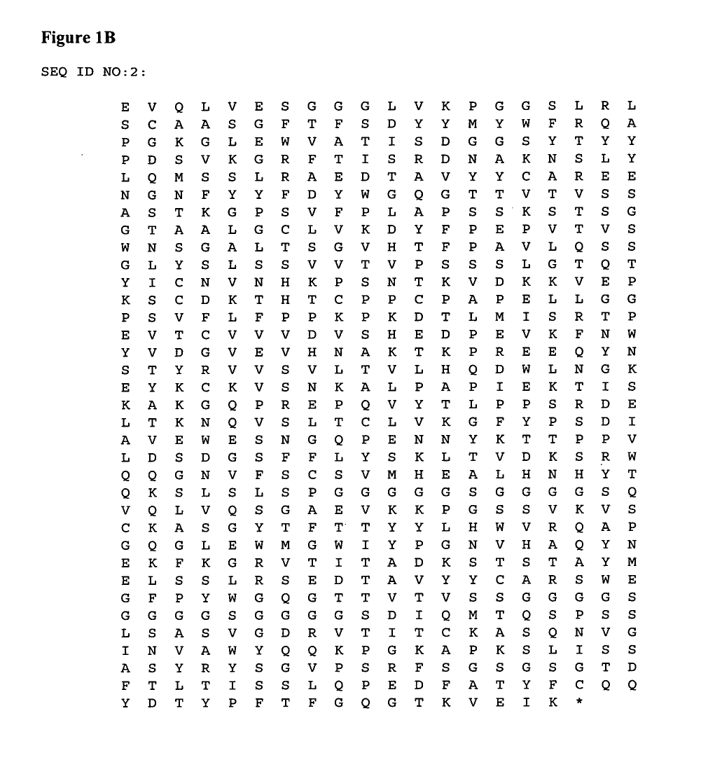

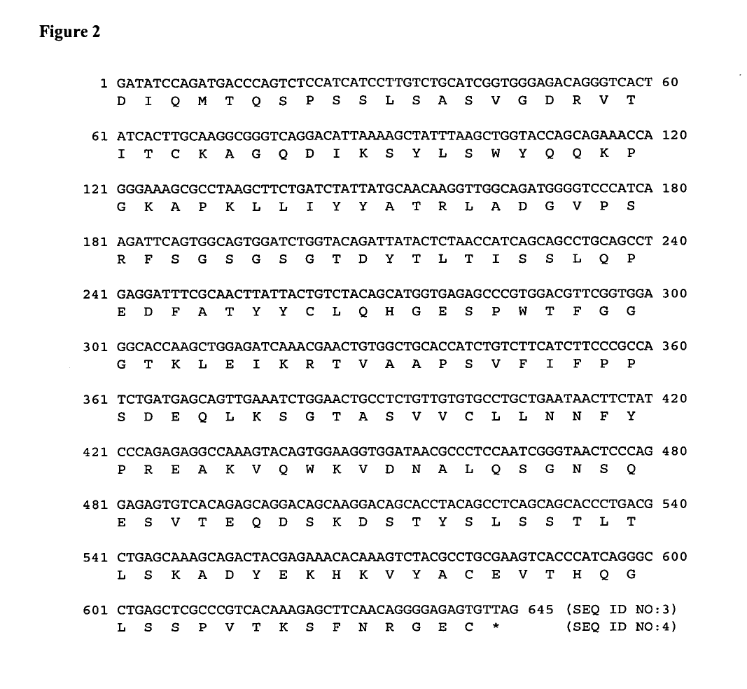

The huCBE11/huBHA10 Bispecific-1 heavy chain was constructed by subcloning the 1087 bp BsrGI-NotI scFv-containing fragment from pXW018 and the 1170 bp NotI-BsrG1 huCBE11 heavy chain fragment from pEAG1325 into the NotI site of the Invitrogen pCEP4-derived EBV expression vector pCH274, producing plasmid pXW020. The DNA sequence of the 2.26 kb NotI insert in pXW020 was confirmed. The cDNA sequence of the mature Bispecific-1 heavy chain is shown in FIG. 1A, and its encoded amino acid sequence in FIG. 1B: it contains the huCBE11 heavy chain with the huBHA10 scFv linked to its C-terminus by a 2× Gly4Ser flexible linker. The huCBE11/huBHA10 Bispecific-1 antibody was transiently expressed in 293-EBNA cells by co-transfection with pXW020 and pAND076, the EBV expression vector for the huCBE11 light chain. The cDNA and encoded amino acid sequences of the mature Bispecific-1 light chain encoded by pAND076 are shown in FIGS. 2A and 2B. The Bispecific-1 construct is schematically depicted in FIG. 6A. Expression of the huCBE11/huBHA10 Bispecific-1 antibody construct was confirmed by Western blot analysis of conditioned medium harvested from transiently transfected cells. A hetero-tetrameric antibody was detected when Western blots were probed with anti-human IgG (heavy plus light)-specific antibodies (parental huBHA10, produced by co-transfection of the EBV expression vectors pKJS046 and pKJS049, parental huCBE11, produced by co-transfection of the EBV expression vectors pAND076 and pAND090 served as the positive controls and empty vector pCH274 served as the negative control). Specificity of the huCBE11/huBHA10 Bispecific-1 antibody construct was confirmed by its ability to stain surface LT-β-Ron both HT29 and COS7 cells, assayed by flow cytometry. A large-scale co-transfection of 293-EBNA cells with pXW020 and pAND076 was prepared to generate antibody for purification.

### Example 2

### Construction of scFv.huCBE11

The first step in construction of the single chain Fv of huCBE11 (scFv.huCBE11) was subcloning the 437 bp NotI-HindIII fragment carrying the huCBE11 heavy chain variable domain from the EBV expression pAND090 into the 2.91 kb NotI-HindIII vector backbone fragment of the pBluescriptIISK+ cloning vector, to make a mutagenesis template called pXW022. The pUC-based plasmid pAND074 carrying the huCBE11 light chain variable domain was subjected to site-directed mutagenesis using a Stratagene QuikChange Mutagenesis kit following the manufacturer's recommended protocol with the primers (a): 5′ CAA TCT CAA AGC TAC CAT GGA GGT CAC CGT CTC CTC TGG GGG CGG GGG GTC CGG GGG AGG CGG GTC GGG AGG TGG CGG AAG TGA TAT CCA GAT GAC CCA G 3′ (SEQ ID NO: 11) and its reverse complement, to add an in-frame BstEII site, 5 heavy chain FR4 residues, and a 3× flexible Gly4Ser linker to the mature N-terminus of the huCBE11 light chain, and (b): 5′ GCA CCA AGC TGG AGA TCA AAG GGG GTG GTG GTT CAG GAG GTG GAG GAT CCT TCC CAC CAT CCA GTG AC 3′ (SEQ ID NO: 12) and its reverse complement, to add an in-frame 2× Gly4Ser linker to the C-terminus of the huCBE11 chain variable domain, with a BamHI site at the 3′ end of the linker's second Gly4Ser. Mutant light chain plasmids containing both the 5′ and 3′ linkers were identified by screening for introduced BstEII and BamHI sites and loss of a BglII restriction sites. The DNA sequence of the 405 bp BstEII-BamHI linkered huCBE11 light chain insert in the resultant plasmid pXW024 was confirmed. The 412 bp NotI-BstEII huCBE11 heavy chain variable domain fragment from pXW022 and the 405 bp BstEII-BamHI linkered huCBE11 light chain fragment from pXW024 were subcloned into the 2.94 kb NotI-BamHI vector backbone fragment the pBluescriptIISK+ cloning vector, to make pXW025. The DNA sequence of the 817 bp NotI-BamHI scFv.huCBE11 insert in pXW025 was confirmed: it contains the heavy chain variable domain (with its signal sequence) linked to the light chain variable domain by a 3× Gly4Ser flexible linker, with a 2× Gly4Ser linker fused to the C-terminus of the light chain variable domain.

### Example 3

### Construction and Expression of scFv.huCBE11-Fc Fusion

To confirm that the scFv.huCBE11 preserved the LTβR binding activity of the parent huCBE11 mAb, a soluble fusion protein was constructed in which the 2× Gly4Ser-linkered scFv.huCBE11 was attached to the N-terminus of a soluble human IgG1 Fc (scFv.huCBE11-Fc). This construct was also expressed accordingly. The plasmid pEAG1397, which contains a recombinant soluble human IgG1 Fc cDNA similar to that described by Lo et al., 1998, Protein Engineering 11: 495-500, was subjected to Quikchange site-directed mutagenesis with the primer 5′ GTT CTG GAT TCC GGC GTC GGG ATC CGA GCC CAA ATC TAG TGA CAA G 3′ (SEQ ID NO:13:) and its reverse complement, to add an in-frame BamHI site at the 5′ end of the hinge. Mutated plasmids were identified by screening for the introduced BamHI site. The DNA sequence of the 711 bp BamHI-NotI Fc fragment of the resultant plasmid pXW023 was confirmed. The 817 bp NotI-BamHI scFv.huCBE11 fragment from pXW025 and the 711 bp BamHI-NotI Fc fragment of pXW023 were subcloned into the NotI site of the Invitrogen pCEP4-derived derived EBV expression vector pCH274, producing plasmid pXW026. The DNA sequence of the 1.53 kb NotI scFv.huCBE11-Fc cDNA insert in the expression vector pXW026 was confirmed. Plasmid pXW026 was transiently transfected into 293-EBNA cells. Expression of the scFv.huCBE11-Fc fusion protein was confirmed by Western blot analysis of conditioned medium harvested from transiently transfected cells. A homodimeric fusion protein of the expected size was detected when Western blots were probed with anti-human Fc-specific antibodies (parental huCBE11, produced by co-transfection of the EBV expression vectors pAND076 and pAND090, served as the positive control and empty vector pCH274 served as the negative control). Specificity of the scFv.huCBE11-Fc was confirmed by its ability to stain surface LTbR on HT29 cells, assayed by flow cytometry. A large-scale transfection of 293-EBNA cells with pXW026 was prepared to generate antibody for purification.

### Example 4

### Construction and Expression of huCBE11/huBHA10 Bispecific-2 Antibody

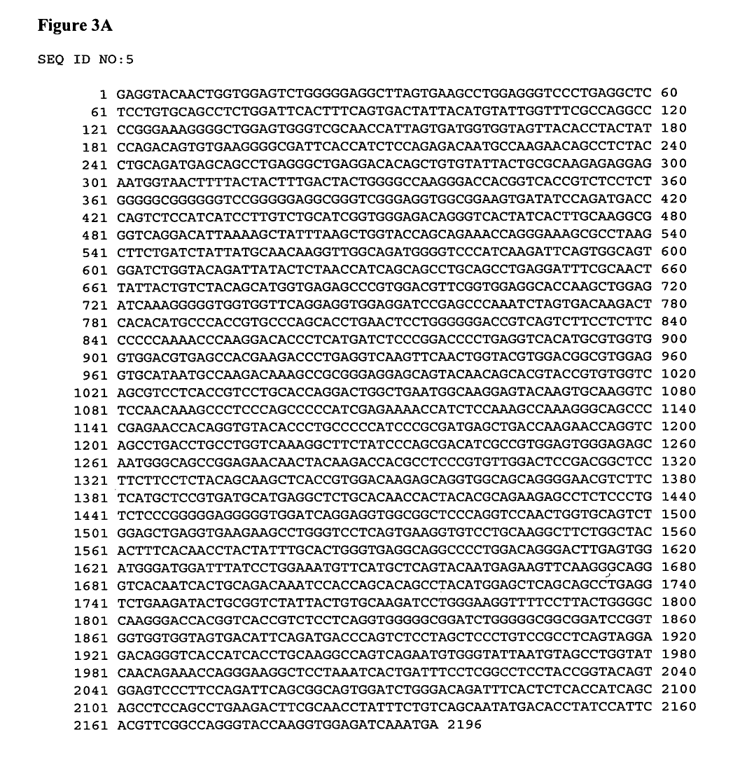

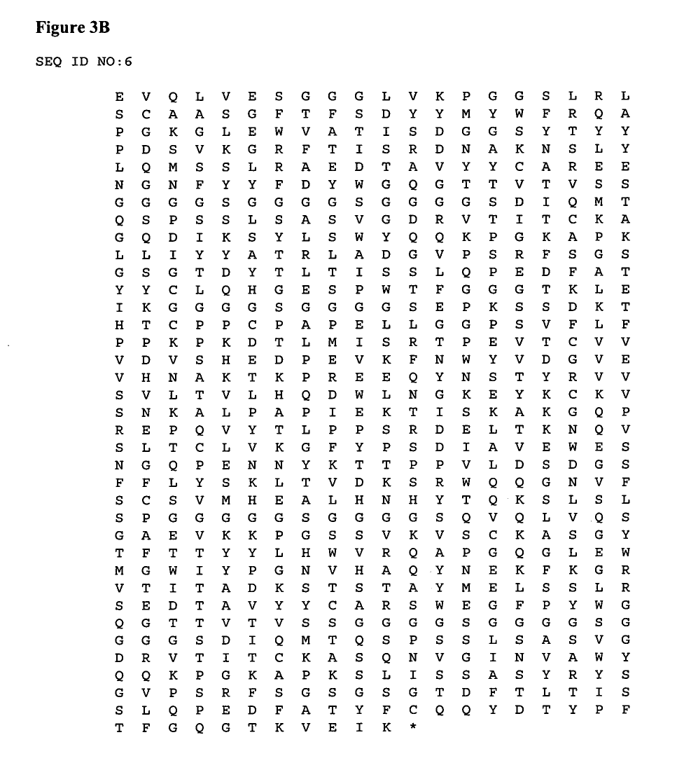

Following the demonstrated expression and LTβR binding by both the Fc-scFv.huBHA10 and scFv.huCBE11-Fc fusion proteins, a combined single fusion protein containing both single scFv entities, called huCBE11/huBHA10 Bispecific-2, was constructed. The huCBE11/huBHA10 Bispecific-2 antibody expression vector was constructed by subcloning the 1087 bp BsrGI-NotI Fc-scFv.huBHA10-containing fragment from pXW018 and the 1220 bp NotI-BsrG1 scFv.huCBE11-Fc fragment from pXW026 into the NotI site of the Invitrogen pCEP4-derived EBV expression vector pCH274, producing plasmid pXW027. The DNA sequence of the 2.31 kb NotI insert in pXW027 was confirmed. The cDNA and amino acid sequences of the mature huCBE11/huBHA10 Bispecific-2 antibody construct encoded by pXW027 are shown in FIGS. 3A and 3B: it contains the scFv.huCBE11 linked at its C-terminus by a 2× Gly4Ser flexible linker fused to a human IgG1 Fc linked at the Fc C-terminus by a 2× Gly4Ser flexible linker fused to the scFv.huBHA10. A schematic of the Bispecific-2 antibody is shown in FIG. 6A. The huCBE11/huBHA10 Bispecific-2 antibody construct was transiently expressed in 293-EBNA cells by transfection with pXW027. Expression of the huCBE11/huBHA10 Bispecific-2 antibody construct was confirmed by Western blot analysis of conditioned medium harvested from transiently transfected cells. A homodimeric antibody was detected when Western blots were probed with anti-human Fc-specific antibodies (Fc-scFv.huBHA10, produced by transfection of the EBV expression vector pXW018, scFv.huCBE11-Fc, produced by transfection of the EBV expression vectors pXW026, served as the positive controls and empty vector pCH274 served as the negative control). Specificity of the huCBE11/huBHA10 Bispecific-2 antibody construct was confirmed by its ability to stain surface LTbR on both HT29 and COS7 cells, assayed by flow cytometry. A large-scale transfection of 293-EBNA cells with pXW027 was prepared to generate antibody for purification.

### Example 5

### Construction and Expression of Monospecific-1 Tetravalent CBE11 Antibodies

The Monospecific antibody constructs similar in design to huCBE11/huBHA10 Bispecific-1 and 2 antibodies were designed with four huCBE11-derived antigen-binding sites. A schematic representation of the CBE11 tetravalent monospecific antibodies is shown in FIG. 6B. Construction of the tetravalent CBE11 antibody required re-engineering the scFv.huCBE11 for fusion to the C-terminus of the Fc. The first step in re-engineering the scFv.huCBE11 encoded by template pXW025 was Stratagene Quikchange site-directed mutagenesis with the mutagenic primers (a): 5′ GGA CTG GAC CTG GAG GGT CCC CGG GGG GGG AGG TGG ATC AGG AGG TGG CGG CTC CGA GGT ACA ACT GGT GG 3′ (SEQ ID NO: 14) and its reverse complement, which adds an in-frame XmaI site followed by a flexible 2× Gly4Ser linker at the 5′ end of the huCBE11 scFv, and (b): 5′ CAT GTA TTG GTT TCG CCA GGC ACC GGG AAA GGG GCT GGA G 3′ (SEQ ID NO: 15) and its reverse complement, to remove an internal XmaI site in FR2 of the huCBE11 heavy chain variable domain. Mutated plasmids were screened for loss of the internal XmaI site and gain of the new XmaI site in the appropriate location. The DNA sequence of the 786 bp BamHI-XmaI huCBE11 scFv insert in the resultant plasmid pXW032 was confirmed. Template pXW032 was subjected to was Stratagene Quikchange site-directed mutagenesis with the mutagenic primer: 5′ GCA CCA AGC TGG AGA TCA AAT GAG GCG GCC GCT CAG GAG GTG GAG GAT CC 3′ (SEQ ID NO: 16) and its reverse complement, to add a termination codon at the end of the scFv's light chain variable domain FR4 and add a 3′ NotI cloning site. Mutated plasmids were screened for gain of a NotI site. The DNA sequence of the 767 bp XmaI-NotI linkered scFv.huCBE11 insert in the resultant plasmid pXW035 was confirmed. The 752 bp NotI-XmaI soluble huIgG1 Fc fragment from pEAG1397 and the 767 bp XmaI-NotI linkered scFv.huCBE11 fragment from pXW035 were subcloned into the NotI site of the pCEP4-derived EBV expression vector pCH274, producing pXW038. The DNA sequence of the 1.52 kb NotI Fc-scFv.huCBE11 insert in pXW038 was confirmed. The soluble Fc-scFv.huCBE11 can be expressed by transient transfection of 293-EBNA cells with pXW038.

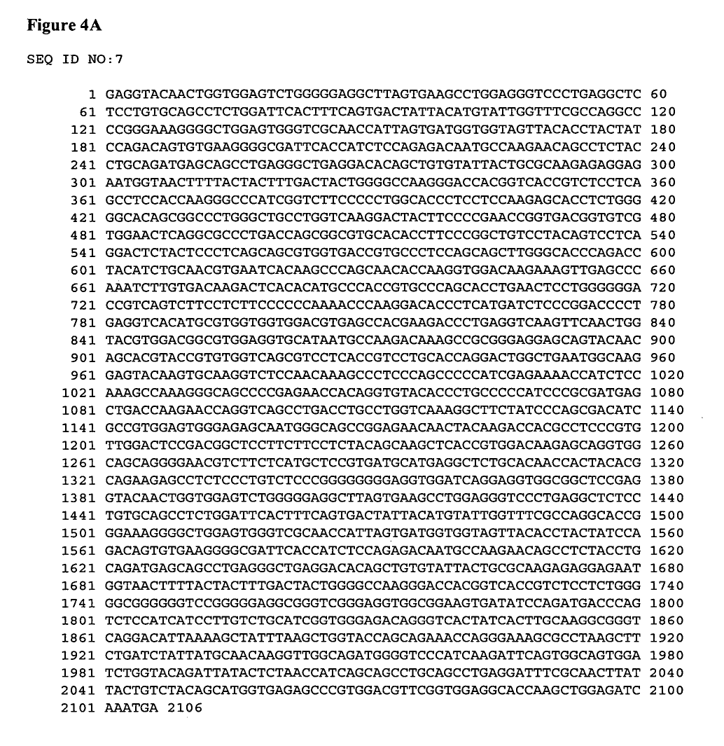

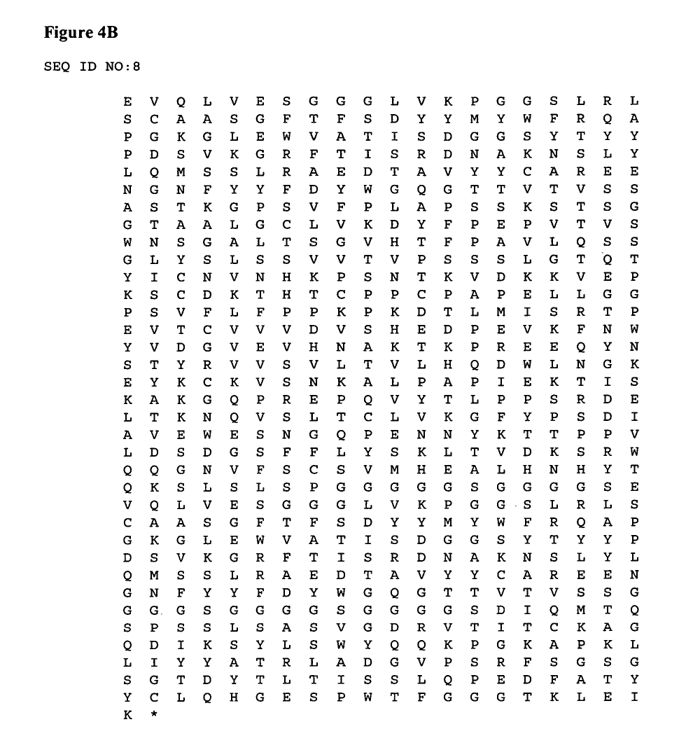

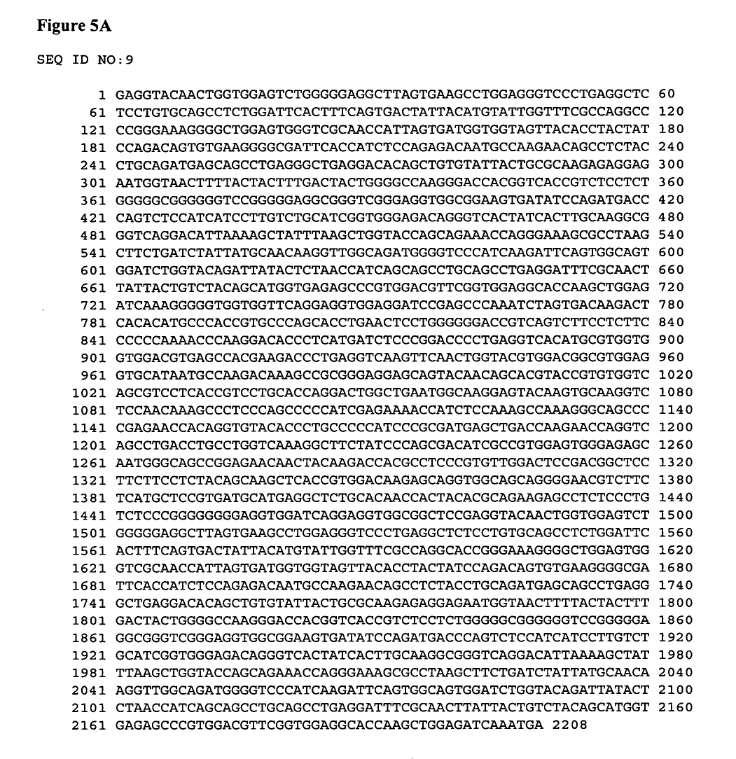

The 1170 bp NotI-BsrGI huCBE11 heavy chain fragment from pEAG1325 and the 1057 bp BsrGI-NotI Fc-scFv.huCBE11 fragment from pXW038 were subcloned into the NotI site of the pCEP4-derived EBV expression vector pCH274, producing pXW039. The DNA sequence of the 2.23 kb NotI insert in pXW039 was confirmed: it contains the huCBE11 heavy chain with the huCBE11 scFv linked to its C-terminus by a 2× Gly4Ser flexible linker. The Monospecific-1 huCBE11 can be expressed by transient co-transfection of 293-EBNA cells with pXW039 and pAND076, the EBV expression vector for the huCBE11 light chain. The DNA and amino acid sequences of the mature heavy chain of the huCBE11 Monospecific-1 antibody construct are depicted in FIGS. 4A and 4B, as well as FIG. 6B

### Example 6

### Construction and Expression of Monospecific-2 Tetravalent huCBE11 Antibody

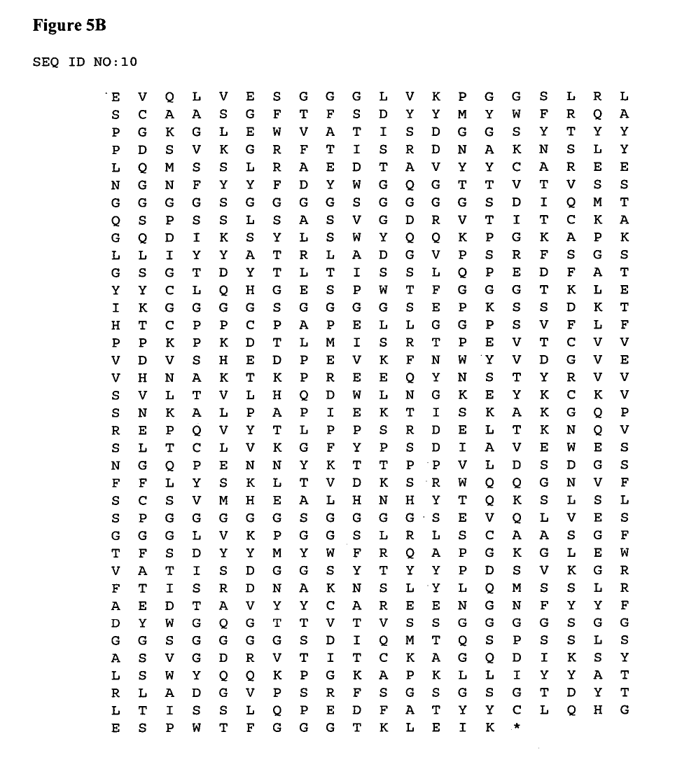

A monospecific tetravalent huCBE11 antibody with a structure similar to that of the huCBE11/huBHA10 Bispecific-2 antibody was constructed. A schematic representation of the CBE11 tetravalent monospecific antibodies is shown in FIG. 6B. Construction was performed according to the following cloning procedures. The 1220 bp NotI-BsrGI Fc-scFv.huCBE11 fragment from pXW026 and the 1057 bp BsrGI-NotI Fc-scFv.huCBE11 fragment from pXW038 were subcloned into the NotI site of the pCEP4-derived EBV expression vector pCH274, producing pXW040. The DNA sequence of the 2.28 kb NotI insert in pXW040 was confirmed: it contains the scFv.huCBE11 linked at its C-terminus by a 2× Gly4Ser flexible linker fused to a human IgG1 Fc linked at the Fc C-terminus by a 2× Gly4Ser flexible linker fused to the scFv.huCBE11. The DNA and amino acid sequences of the mature Monospecific-2 huCBE11 construct encoded by pXW040 are shown in FIGS. 5A and 5B, and schematically in FIG. 6B. The Monospecific-2 antibody construct can be transiently expressed in 293-EBNA cells by transfection with pXW040.

### Example 7

### Construction and Expression of Pentameric Chimeric CBE11

Cloning of the CBE11 variable domains and construction of EBV expression vectors pEAG982 and pEAG983 for chimeric CBE11 (chCBE11) kappa light and IgG1 heavy chains, respectively, was previously described in Applicant's co-pending application PCT publication no. WO 02/30986, incorporated by reference herein.

Smith et al. (1995) J. Immunol. 154: 2226 reported that the addition of the C-terminal IgM tailpiece to IgG constant regions could produce polymeric recombinant IgM-like antibodies, greatly increasing their avidities. The 18-amino acid C-terminal tailpiece from IgM was added to the C-terminus of the chimeric CBE11-huIgG1 heavy chain by site-directed mutagenesis, duplicating the C-terminal tailpiece described by Smith et al., in which the wildtype IgG1 C-terminus PGK sequence is substituted by the human IgM C-terminal sequence TGK PTLYNVSLVM SDTAGTCY (SEQ ID NO: 21). The template pEAG409, which contains the human IgG1 Fc cDNA as a SalI-NotI fragment in a pUC-derived cloning vector was subjected to unique site elimination (USE) mutagenesis using an Amersham Pharmacia Biotech USE mutagenesis kit following the manufacturer's recommended protocol using the mutagenic primer 5′ GAA GAG CCT CTC CCT GTC TAC CGG GAA ACC CAC CCT GTA CAA CGT GTC CCT GTG AGT GCG GCG GCC GCC 3′ (SEQ ID NO: 22), which mutated proline 445 (Kabat EU numbering) to threonine and added the first 8 amino acids of the IgM tailpiece. Mutated plasmids were identified by screening for introduced RsaI and AflIII sites. The DNA sequence of the Nsi-NotI insert containing the C-terminus of the IgG cDNA in the resultant plasmid pEAG423 was confirmed.

Template plasmid pEAG423 was subjected to another round of USE mutagenesis using the mutagenic primer 5′ CCC TGT ACA ACG TGT CCC TGG TCA TGT CCG ACA CAG CTG GCA CCT GCT ACT GAG TGC GGC GGC CGC C 3′ (SEQ ID NO: 23), which added the last 10 amino acids of the IgM tailpiece. Mutated plasmids were identified by screening for introduced DdeI and PvuII sites. The Fc cDNA sequence in the resultant plasmid pEAG427 was confirmed. To add the IgM tailpiece to the C-terminus of the chimeric CBE11-IgG1 heavy chain, the 1.57 kb NotI-NsiI fragment from pEAG983 and the 0.12 kb NsiI-NotI fragment from pEAG427 were subcloned into the NotI site of the pCEP4 (Invitrogen) derived EBV expression vector pCH269, producing plasmid pEAG995.

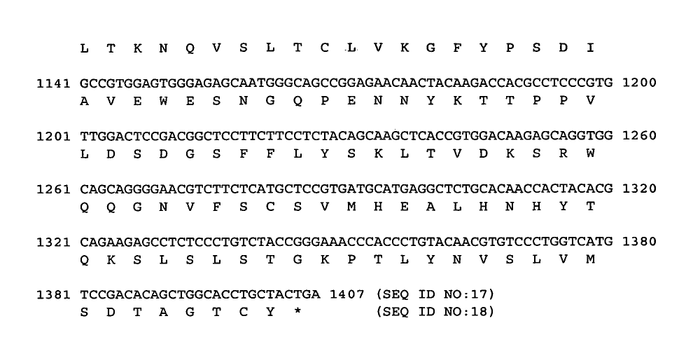

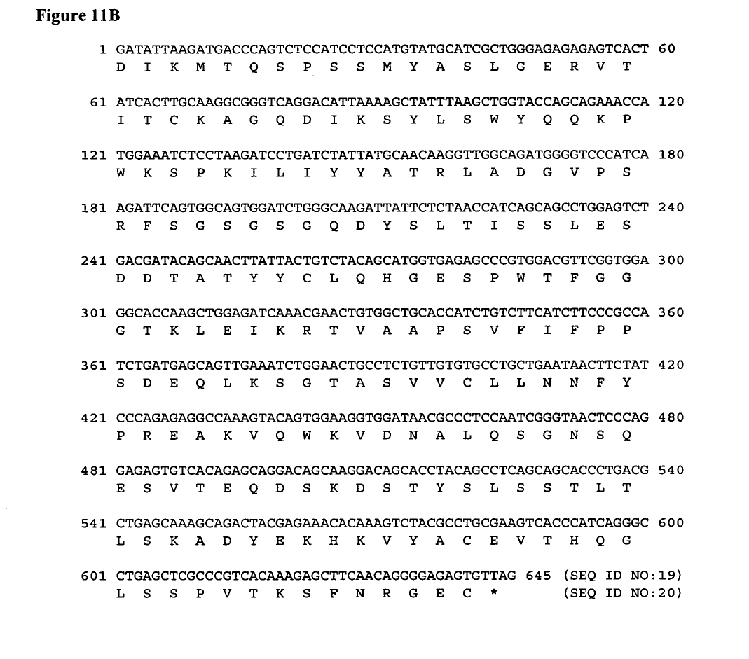

Pentameric chimeric CBE11 antibody was produced by transient co-transfection of 293-EBNA cells with heavy chain vector pEAG995 and light chain vector pEAG982. The predicted mature cDNA sequences encoded by pEAG995 and pEAG982 are shown in FIG. 11. Transfected cells secreted both monomeric and pentameric chCBE11, with a greatly enhanced avidity. Western blot analysis indicated that the heavy chain produced in co-transfections with pEAG995 had a larger size than that produced in co-transfection with the wildtype chCBE11-IgG1 heavy chain vector pEAG983. The transient co-transfection with pEAG982 and pEAG995 was scaled up to generate pentameric antibody for purification.

### Example 8

### In Vitro Analysis of Multivalent Anti-LTBR Antibodies

In order to determine the in vitro efficacy of the multivalent antibodies of the invention at inhibiting tumor cell growth, e.g., Bispecific-1, Bispecific-2, Monospecific 1, and Monospecific 2 antibody, the multivalent antibodies were tested in parallel versus various forms of CBE11 and BHA10 antibodies.

Antibodies were prepared according to the following procedures. Humanized CBE11 and humanized BHA10 antibodies were generated from stable expression CHO cell lines. Bispecific 1, Bispecific 2, Monospecific 1, Monospecific 2 and the chimeric CBE11 pentamer antibodies were generated from transient expression in EBNA293 cells. All seven antibodies were first purified by chromatography on protein A Sepharose (Amersham-Pharmacia). Humanized CBE11 was further purified by chromatography on Fractogel Hicap TMAE (EM Industries) and Phenyl Sepharose (Amersham-Pharmacia). Humanized BHA10 and Bispecific-1 antibodies were further purified by chromatography on Fractogel Hicap SE (EM Industries). The chimeric CBE11 pentamer was further purified by size exclusion chromatography on Sepharose 6B.

A small amount of each antibody (except chimeric CBE11 pentamer) was further purified by size exclusion chromatography on G3000SW (TosoHaas) to remove any aggregates (dimers and bigger aggregates). An extinction coefficient of 1.4 was used for humanized CBE11, humanized BHA10 and the CBE11 pentamer. An extinction coefficient of 1.6 was used for Bispecific-1 and Monospecific 1. An extinction coefficient of 1.7 was used for Bispecific 2 and Monospecific 2. SDS-PAGE and mass spectrometry analyses indicated that the purified antibodies were at least 95% intact.

To determine the anti-tumor activity of the multivalent antibodies of the invention, each antibody was assayed for its ability to inhibit tumor cell growth in vitro using the HT29 adenocarcinoma cell line. Colon adenocarcinoma cell line HT-29 (ATCC) were grown in MEM Earle's supplemented with 10% Fetal Bovine Serum, 2 mM Glutamine, 1 mM sodium pyruvate, 1% NEAA at 37° C. in 5% CO₂. HT29 cells were seeded in the wells of 96-well plates (5,000 cells/well) in medium containing human interferon gamma (80 U/ml) and various concentrations of antibody agents. After 4 days in culture, living cells were stained with MTT [3,4,5-dimethylthiazol-2-yl) 2,5 diphenyltetrazolium bromide] which is reduced in the mitochondria to a colored formazan product absorbing at 570 nm.

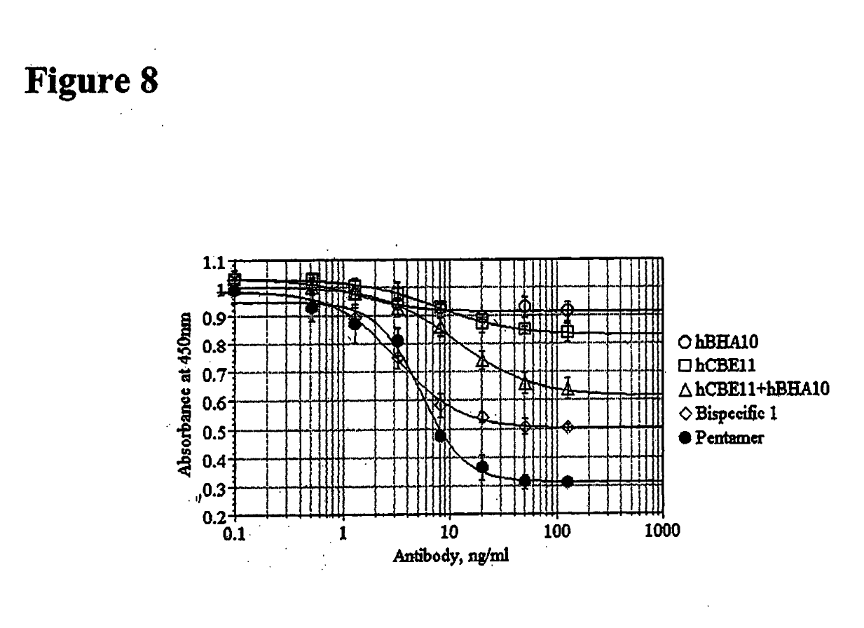

The growth of HT29 adenocarcinoma cell line was reduced to various degrees in presence of the various anti-LTBR agonist antibodies (and interferon gamma), as shown in FIG. 8. Each anti-LTBR agent reached a different plateau corresponding to a maximum of activity (minimum absorbance value at 450 nm). This plateau was a measure of the LTBR activating agent's potency. Humanized BHA10 was the least effective agent (minimum A450=0.9), followed by humanized CBE11 (minimum A450=0.8). Pairing humanized CBE11 and humanized BHA10 increases significantly the efficacy (minimum A450=0.6) but combining huBHA10 antigen-binding regions to huCBE11 in a single tetravalent molecule (Bispecific 1) increased the efficacy even more (minimum A450=0.5). CBE11 pentamer with its ten antigen-binding sites was the most potent agent (minimum A450=0.3). In sum, the bispecific (BHA10/CBE11) and monospecific (CBE11) tetravalent antibodies showed an increased ability to inhibit tumor cell growth in comparison to huCBE11 and huBHA10.

### Example 9

### Comparative Response of the WiDr Human Colorectal Adenocarcinoma to huBHA10 and Bispecific 1 and 2 in Athymic Nude Mice

The WiDr human colorectal adenocarcinoma in-vitro cell line was obtained from the American Tissue Type Collection. The cell line was passed in vitro for 4 passages in Minimum essential medium Eagle with 2 mM L-glutamine and Earle's BSS adjusted to contain 1.5 g/L sodium bicarbonate, 0.1 mM non-essential amino acids, and 1.0 mM sodium pyruvate, 90%; fetal bovine serum, 10% without antibiotics.

220 athymic nude mice females were obtained from Harlan Sprague Dawley (Madison, Wis.). The animals were individually marked by ear punches prior to randomization. On the day of randomization into test and control groups animals were implanted with BioMedic animal ID chips. The animals were implanted with the cell inoculum of 2×10E6 cells/mouse (RPMI-1640 w/o serum) subcutaneously in the right flank area. At day 5 post-implantation, tumor size measurements were recorded and measurements continued either daily or every other day until staging at day 7 post-implantation.

Mice were selected with tumors measuring a minimum size of 5 mm (length)×5 mm (width), determined using vernier calipers. Mice were randomize into test and control group using LabCat software. Initial body weights were recorded and treatments administered to treatment groups as follows:

TABLE 1

| | | Number of |
|---|---|---|
| Group | Treatment Regimen | Animals |
| PBS (non- | 200 uL/mouse, i.p., 3×/week × 4 wks. | 20 |
| pyrogenated) | (M, W, F) | |
| Control, | | |
| Taxol | 25.0 mg/kg/inj., i.p., Q4DX3 | 8 |
| huBHA10 | 200 ug/200 uL/inj., i.p, 2×/week × 4 wks. | 8 |
| | (M, Th) | |
| huBHA10 | 100 ug/200 uL/inj., i.p, 2×/week × 4 wks. | 8 |
| | (M, Th) | |
| huBHA10 | 50 ug/200 uL/inj., i.p, 2×/week × 4 wks. | 8 |
| | (M, Th) | |
| huBHA10 | 25 ug/200 uL/inj., i.p, 2×/week × 4 wks. | 8 |
| | (M, Th) | |
| Bispecific 1 | 200 ug/200 uL/inj., i.p, 3×/week × 4 wks. | 8 |
| | (M, W, F) | |
| Bispecific 1 | 100 ug/200 uL/inj., i.p, 3×/week × 4 wks. | 8 |
| | (M, W, F) | |
| Bispecific 1 | 50 ug/200 uL/inj., i.p, 3×/week × 4 wks. | 8 |
| | (M, W, F) | |
| Bispecific 1 | 25 ug/200 uL/inj., i.p, 3×/week × 4 wks. | 8 |
| | (M, W, F) | |

The effect of treatment was assessed by the follwing indicators: initial body weight, tumor size and body weight measurements twice weekly, serum samples (retro-orbital bleed) of huBHA10 groups on days 14, 24, 35 (treatment days 7, 17, 28), and serum samples (retro-orbital bleed) of BS1 groups and 10 mice from vehicle control group on days 13, 23, 34 (treatment days 7, 18, 28).

The response of the WiDr human colon adenocarcinoma tumor to Bispecific-1 is depicted in FIG. 9. A comparison of the efficacy of Bispecific-1 and huCBE11 in inhibiting WiDr human colon adenocarcinoma tumor growth is depicted in FIG. 10.

### EQUIVALENTS

The present invention provides among other things novel antibody constructs. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entirety as if each individual publication or patent was specifically and individually indicated to be incorporated by reference. In case of conflict, the present application, including any definitions herein, will control.

We claim:

1. A multivalent antibody comprising at least one antigen recognition site specific for a lymphotoxin-beta receptor (LT-β-R) epitope.

2. The multivalent antibody of claim 1, wherein at least one antigen recognition site is located on a scFv domain.

3. The multivalent antibody of claim 1, wherein all antigen recognition sites are located on scFv domains.

4. The multivalent antibody of claim 1, wherein the antibody construct is monospecific.

5. The multivalent antibody of claim 4, wherein the antibody construct is specific for the epitope to which CBE11 binds.

6. The multivalent antibody of claim 5, wherein the antibody construct is tetravalent.

7. The multivalent antibody of claim 4, wherein the antibody construct is specific for the BHA10 epitope.

8. The multivalent antibody of claim 7, wherein the antibody construct is tetravalent.

9. The multivalent antibody of claim 4, wherein at least one antigen recognition site is located on a scFv domain.

10. The multivalent antibody of claim 4, wherein all antigen recognition sites are located on scFv domains.

11. The multivalent antibody of claim 1, wherein the antibody construct is bispecific.

12. The multivalent antibody of claim 11, wherein the antibody construct is specific for at least two members of the group of lymphotoxin-beta receptor (LT-β-R) epitopes consisting of: BKA11, CDH10, BCG6, AGH1, BDA8, CBE11 and BHA10.

13. The multivalent antibody of claim 12, wherein the antibody construct is specific for the CBE11 and BHA10 epitopes.

14. The multivalent antibody of claim 13, wherein the antibody construct is tetravalent.

15. The multivalent antibody of claim 14, wherein the antibody construct has two CBE11-specific antigen recognition sites and two BHA10-specific recognition sites.

16. The multivalent antibody of claim 11, wherein at least one antigen recognition site is located on a scFv domain.

17. The multivalent antibody of claim 11, wherein all antigen recognition sites are located on scFv domains.

18. A method of treating cancer comprising administering a multivalent antibody comprising at least one antigen recognition site specific for a lymphotoxin-beta receptor (LT-β-R) epitope.

19. The method of claim 18, wherein the antibody construct is monospecific.

20. The method of claim 19, wherein the antibody construct is specific for the epitope to which CBE11 binds.

21. The method of claim 20, wherein the antibody construct is tetravalent.

22. The method of claim 18, wherein the antibody construct is bispecific.

23. The method of claim 22, wherein the antibody construct is specific for at least two members of the group of lymphotoxin-beta receptor (LT-β-R) epitopes consisting of: BKA11, CDH10, BCG6, AGH1, BDA8, CBE11 and BHA10.

24. The method of claim 22, wherein the antibody construct is specific for the CBE11 and BHA10 epitopes.

25. The method of claim 24, wherein the antibody construct is tetravalent.

26. The method of claim 18, wherein said subject is human.

27. A pharmaceutical composition comprising an effective amount of a multivalent antibody and a pharmaceutically acceptable carrier, wherein the multivalent antibody comprises at least one antigen recognition site specific for a lymphotoxin-beta receptor (LT-β-R) epitope.

28. The composition of claim 27, wherein the antibody construct is monospecific.

29. The composition of claim 28, wherein the antibody construct is specific for the epitope to which CBE11 binds.

30. The composition of claim 29, wherein the antibody construct is tetravalent.

31. The composition of claim 27, wherein the antibody construct is bispecific.

32. The composition of claim 31, wherein the antibody construct is specific for at least two members of the group of lymphotoxin-beta receptor (LT-β-R) epitopes consisting of: BKA11, CDH10, BCG6, AGH1, BDA8, CBE11 and BHA10.

33. The composition of claim 31, wherein the antibody construct is specific for the CBE11 and BHA10 epitopes.

34. The composition of claim 33, wherein the antibody construct is tetravalent.

35. A nucleic acid comprising a DNA sequence selected from the group consisting of SEQ ID No.: 1; SEQ ID No.: 3; SEQ ID No.: 5; SEQ ID No.: 7; and SEQ ID No.: 9.

36. An expression vector comprising the nucleic acid of claim 35.

37. A cell comprising the expression vector of claim 36.

38. A polypeptide comprising an amino acid sequence selected from the group consisting of SEQ ID No: 2; SEQ ID No.: 4; SEQ ID No.: 6; SEQ ID No.: 8; and SEQ ID No.: 10.


SEQUENCE LISTING (23 sequences)

SEQ ID NO: 1 - DNA, 2094 bp, Artificial Sequence
  Description of Artificial Sequence Heavy chain of huCBE11/huBHA10 bispecific-1 antibody construct
gaggtacaactggtggagtctgggggaggcttagtgaagcctggagggtccctgaggctctcctgtgcagcctctggattcactttcagtgactattacatgtattggtttcgccaggccccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggggagggggtggatcaggaggtggcggctcccaggtccaactggtgcagtctggagctgaggtgaagaagcctgggtcctcagtgaaggtgtcctgcaaggcttctggctacactttcacaacctactatttgcactgggtgaggcaggcccctggacagggacttgagtggatgggatggatttatcctggaaatgttcatgctcagtacaatgagaagttcaagggcagggtcacaatcactgcagacaaatccaccagcacagcctacatggagctcagcagcctgaggtctgaagatactgcggtctattactgtgcaagatcctgggaaggttttccttactggggccaagggaccacggtcaccgtctcctcaggtgggggcggatctgggggcggcggatccggtggtggtggtagtgacattcagatgacccagtctcctagctccctgtccgcctcagtaggagacagggtcaccatcacctgcaaggccagtcagaatgtgggtattaatgtagcctggtatcaacagaaaccagggaaggctcctaaatcactgatttcctcggcctcctaccggtacagtggagtcccttccagattcagcggcagtggatctgggacagatttcactctcaccatcagcagcctccagcctgaagacttcgcaacctatttctgtcagcaatatgacacctatccattcacgttcggccagggtaccaaggtggagatcaaatga

SEQ ID NO: 2 - protein, 697 aa, Artificial Sequence
  Description of Artificial Sequence Heavy chain of huCBE11/huBHA10 bispecific-1 antibody construct
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSSGGGGSGGGGSGGGGSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIK

SEQ ID NO: 3 - DNA, 645 bp, Artificial Sequence
  Description of Artificial Sequence Light chain of huCBE11/huBHA10 bispecific-1 antibody construct
gatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 4 - protein, 214 aa, Artificial Sequence
  Description of Artificial Sequence Light chain of huCBE11/huBHA10 bispecific-1 antibody construct
DIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 2196 bp, Artificial Sequence
  Description of Artificial Sequence huCBE11/huBHA10 bispecific-2 antibody construct
gaggtacaactggtggagtctgggggaggcttagtgaagcctggagggtccctgaggctctcctgtgcagcctctggattcactttcagtgactattacatgtattggtttcgccaggccccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctctgggggcggggggtccgggggaggcgggtcgggaggtggcggaagtgatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaagggggtggtggttcaggaggtggaggatccgagcccaaatctagtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccgggggagggggtggatcaggaggtggcggctcccaggtccaactggtgcagtctggagctgaggtgaagaagcctgggtcctcagtgaaggtgtcctgcaaggcttctggctacactttcacaacctactatttgcactgggtgaggcaggcccctggacagggacttgagtggatgggatggatttatcctggaaatgttcatgctcagtacaatgagaagttcaagggcagggtcacaatcactgcagacaaatccaccagcacagcctacatggagctcagcagcctgaggtctgaagatactgcggtctattactgtgcaagatcctgggaaggttttccttactggggccaagggaccacggtcaccgtctcctcaggtgggggcggatctgggggcggcggatccggtggtggtggtagtgacattcagatgacccagtctcctagctccctgtccgcctcagtaggagacagggtcaccatcacctgcaaggccagtcagaatgtgggtattaatgtagcctggtatcaacagaaaccagggaaggctcctaaatcactgatttcctcggcctcctaccggtacagtggagtcccttccagattcagcggcagtggatctgggacagatttcactctcaccatcagcagcctccagcctgaagacttcgcaacctatttctgtcagcaatatgacacctatccattcacgttcggccagggtaccaaggtggagatcaaatga

SEQ ID NO: 6 - protein, 731 aa, Artificial Sequence
  Description of Artificial Sequence huCBE11/huBHA10 bispecific-2 antibody construct
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSGGGGSGGGGSGGGGSDIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIKGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSQVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSSGGGGSGGGGSGGGGSDIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIK

SEQ ID NO: 7 - DNA, 2106 bp, Artificial Sequence
  Description of Artificial Sequence Heavy chain of huCBE11 monospecific-1 antibody construct
gaggtacaactggtggagtctgggggaggcttagtgaagcctggagggtccctgaggctctcctgtgcagcctctggattcactttcagtgactattacatgtattggtttcgccaggccccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggggggggaggtggatcaggaggtggcggctccgaggtacaactggtggagtctgggggaggcttagtgaagcctggagggtccctgaggctctcctgtgcagcctctggattcactttcagtgactattacatgtattggtttcgccaggcaccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctctgggggcggggggtccgggggaggcgggtcgggaggtggcggaagtgatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaatga

SEQ ID NO: 8 - protein, 701 aa, Artificial Sequence
  Description of Artificial Sequence Heavy chain of huCBE11 monospecific-1 antibody construct
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSGGGGSGGGGSGGGGSDIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIK

SEQ ID NO: 9 - DNA, 2208 bp, Artificial Sequence
  Description of Artificial Sequence huCBE11 monospecific-2 antibody construct
gaggtacaactggtggagtctgggggaggcttagtgaagcctggagggtccctgaggctctcctgtgcagcctctggattcactttcagtgactattacatgtattggtttcgccaggccccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctctgggggcggggggtccgggggaggcgggtcgggaggtggcggaagtgatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaagggggtggtggttcaggaggtggaggatccgagcccaaatctagtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggggggggaggtggatcaggaggtggcggctccgaggtacaactggtggagtctgggggaggcttagtgaagcctggagggtccctgaggctctcctgtgcagcctctggattcactttcagtgactattacatgtattggtttcgccaggcaccgggaaaggggctggagtgggtcgcaaccattagtgatggtggtagttacacctactatccagacagtgtgaaggggcgattcaccatctccagagacaatgccaagaacagcctctacctgcagatgagcagcctgagggctgaggacacagctgtgtattactgcgcaagagaggagaatggtaacttttactactttgactactggggccaagggaccacggtcaccgtctcctctgggggcggggggtccgggggaggcgggtcgggaggtggcggaagtgatatccagatgacccagtctccatcatccttgtctgcatcggtgggagacagggtcactatcacttgcaaggcgggtcaggacattaaaagctatttaagctggtaccagcagaaaccagggaaagcgcctaagcttctgatctattatgcaacaaggttggcagatggggtcccatcaagattcagtggcagtggatctggtacagattatactctaaccatcagcagcctgcagcctgaggatttcgcaacttattactgtctacagcatggtgagagcccgtggacgttcggtggaggcaccaagctggagatcaaatga

SEQ ID NO: 10 - protein, 735 aa, Artificial Sequence [sequence omitted]

SEQ ID NO: 11 - DNA, 100 bp, Artificial Sequence
  Description of Artificial Sequence Synthetic primer
caatctcaaagctaccatggaggtcaccgtctcctctgggggcggggggtccgggggaggcgggtcgggaggtggcggaagtgatatccagatgacccag

SEQ ID NO: 12 - DNA, 68 bp, Artificial Sequence
  Description of Artificial Sequence Synthetic primer
gcaccaagctggagatcaaagggggtggtggttcaggaggtggaggatccttcccaccatccagtgac

SEQ ID NO: 13 - DNA, 46 bp, Artificial Sequence
  Description of Artificial Sequence Synthetic primer
gttctggattccggcgtcgggatccgagcccaaatctagtgacaag

SEQ ID NO: 14 - DNA, 71 bp, Artificial Sequence
  Description of Artificial Sequence Synthetic primer
ggactggacctggagggtccccggggggggaggtggatcaggaggtggcggctccgaggtacaactggtgg

SEQ ID NO: 15 - DNA, 40 bp, Artificial Sequence
  Description of Artificial Sequence Synthetic primer
catgtattggtttcgccaggcaccgggaaaggggctggag

SEQ ID NO: 16 - DNA, 50 bp, Artificial Sequence
  Description of Artificial Sequence Synthetic primer
gcaccaagctggagatcaaatgaggcggccgctcaggaggtggaggatcc

SEQ ID NO: 17 - DNA, 1407 bp, Artificial Sequence
  Description of Artificial Sequence CBE11 pentameric heavy chain antibody construct
gaggtacaactggtggagtctgggggaggcttagtgaagcctggagggtccctgaaactctcctgtgcagcctctggattcactttcagtgactattacatgtattggtttcgccagactccggaaaagaggctggagtgggtcgcaaccattagtgatggtggtagttacacctactatccagacagtgtgaaggggcgattcaccatctccagagacaatgccaagaacaacctgtacctgcaaatgagcagtctgaagtctgaggacacagccatgtattactgtgtaagagaggagaatggtaacttttactactttgactactggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctaccgggaaacccaccctgtacaacgtgtccctggtcatgtccgacacagctggcacctgctactga

SEQ ID NO: 18 - protein, 468 aa, Artificial Sequence
  Description of Artificial Sequence CBE11 pentameric heavy chain antibody construct
EVQLVESGGGLVKPGGSLKLSCAASGFTFSDYYMYWFRQTPEKRLEWVATISDGGSYTYYPDSVKGRFTISRDNAKNNLYLQMSSLKSEDTAMYYCVREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSTGKPTLYNVSLVMSDTAGTCY

SEQ ID NO: 19 - DNA, 645 bp, Artificial Sequence
  Description of Artificial Sequence CBE11 chimeric light chain antibody construct
gatattaagatgacccagtctccatcctccatgtatgcatcgctgggagagagagtcactatcacttgcaaggcgggtcaggacattaaaagctatttaagctggtaccagcagaaaccatggaaatctcctaagatcctgatctattatgcaacaaggttggcagatggggtcccatcaagattcagtggcagtggatctgggcaagattattctctaaccatcagcagcctggagtctgacgatacagcaacttattactgtctacagcatggtgagagcccgtggacgttcggtggaggcaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 214 aa, Artificial Sequence
  Description of Artificial Sequence CBE11 chimeric light chain antibody construct
DIKMTQSPSSMYASLGERVTITCKAGQDIKSYLSWYQQKPWKSPKILIYYATRLADGVPSRFSGSGSGQDYSLTISSLESDDTATYYCLQHGESPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 21 aa, Homo sapiens
TGKPTLYNVSLVMSDTAGTCY

SEQ ID NO: 22 - DNA, 69 bp, Artificial Sequence
  Description of Artificial Sequence Synthetic primer
gaagagcctctccctgtctaccgggaaacccaccctgtacaacgtgtccctgtgagtgcggcggccgcc

SEQ ID NO: 23 - DNA, 67 bp, Artificial Sequence
  Description of Artificial Sequence Synthetic primer
ccctgtacaacgtgtccctggtcatgtccgacacagctggcacctgctactgagtgcggcggccgcc